(19) United States
(12) Patent Application Publication
(10) Pub. No.: US 2026/0297811 A1
(43) Pub. Date: Oct. 1, 2026

(54) STABILIZED FIBRONECTIN BASED SCAFFOLD MOLECULES

(71) Applicant: Bristol-Myers Squibb Company, Princeton, NJ (US)

(72) Inventor: Dasa LIPOVSEK, Pepperell, MA (US)

(21) Appl. No.: 19/425,204

(22) Filed: Dec. 18, 2025

Related U.S. Application Data
(62) Division of application No. 17/867,434, filed on Jul. 18, 2022, now Pat. No. 12,529,165.
Application No. 17/867,434 is a division of application No. 16/560,521, filed on Sep. 4, 2019, now Pat. No. 11,407,815.
Application No. 16/560,521 is a continuation of application No. 15/127,183, filed on Mar. 19, 2015, now Pat. No. 10,450,363.
(60) Provisional application No. 61/955,975, filed on Mar. 20, 2014.
(60) Provisional application No. 62/084,270, filed on Nov. 25, 2014.

(51) Int. Cl. C40B 40/10

(52) U.S. Cl. CPC C40B 40/10; C07K 2318/20

(57) ABSTRACT

Provided herein are proteins comprising a fibronectin based scaffold (FBS) domain, e.g., ¹⁰Fn3 molecules, that bind specifically to a target, and wherein the FBS domain is linked at its C-terminus to a region consisting of PmXn, wherein P is proline, X is any amino acid and wherein n is 0 or an integer that is at least 1 and m is an integer that is at least 1, and wherein the PmXn moiety provides an enhanced property to the FBS domain, e.g., enhanced stability, relative to the protein that is not linked to the PmXn moiety.

### CROSS-REFERENCE TO RELATED APPLICATIONS

This application is a divisional of U.S. patent application Ser. No. 17/867,434 (now U.S. Pat. No. 12,529,165), filed Jul. 18, 2022, which is a divisional of U.S. patent application Ser. No. 16/560,521 (now U.S. Pat. No. 11,407,815), filed Sep. 4, 2019, which is a continuation of U.S. patent application Ser. No. 15/127,183 (now U.S. Pat. No. 10,450,363), filed Sep. 19, 2016, which is a 35 U.S.C. 371 national stage filing of International Application No. PCT/US2015/021466, filed Mar. 19, 2015, which claims the benefit of U.S. Provisional Application No. 61/955,975, filed Mar. 20, 2014, and U.S. Provisional Application No. 62/084,270, filed Nov. 25, 2014. The contents of the aforementioned applications are hereby incorporated by reference.

### REFERENCE TO SEQUENCE LISTING

The instant application contains a Sequence Listing which has been submitted electronically in xml format and is hereby incorporated by reference in its entirety. Said xml copy, created on Apr. 25, 2026 is named MXI-553USCNDV2.xml and is 189,629 bytes in size.

### BACKGROUND

Adnectins are a class of therapeutic proteins with high-affinity and specific target-binding properties that are derived from the tenth human fibronectin type III domain (¹⁰Fn3). Whereas wild-type ¹⁰Fn3 is extremely stable and soluble, target-binding variants of ¹⁰Fn3, which contain in the order of 4-31 mutations from the wild-type sequence, vary widely in stability and solubility. In other words, any mutations from the wild-type ¹⁰Fn3 sequence, even if required for target binding, carries a risk of reducing the stability of protein. As a consequence, it would be desirable to identify modifications that can be made to the wild-type ¹⁰Fn3 sequence that would stabilize it, preferably regardless of the identity of the residues that mediate Adnectin binding to their therapeutic targets.

### SUMMARY

Provided herein are stabilized fibronectin based scaffold (FBS) proteins, e.g., Fn3, such as ¹⁰Fn3 molecules (e.g., human ¹⁰Fn3 molecules) that are linked at their C-terminus to a moiety consisting of the amino acid sequence PmXn, wherein P is proline, X is any amino acid, m is an integer that is at least 1 and n is 0 or an integer that is at least 1, and wherein the PmXn moiety enhances at least one characteristic, e.g., thermostability, of the FBS proteins.

### BRIEF DESCRIPTION OF THE FIGURES

FIG. 1: Representation of a crystal structure of the human ¹⁰Fn3 domain (PDB ID: 1FNA), and the protein sequence of the polypeptide visible in the structure. The last two residues defined in the structure, the “EI” in the sequence of interest, are shown as black spheres, immediately downstream from the C-terminal beta strand, G.

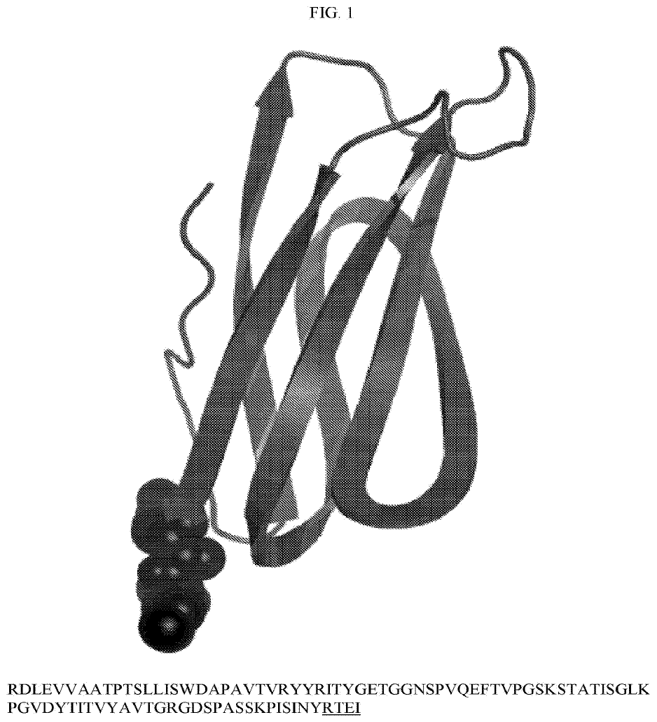

### DETAILED DESCRIPTION

### Definitions

An “amino acid residue” is the remaining portion of an amino acid after a water molecule has been lost (an H+ from the nitrogenous side and an OH— from the carboxylic side) in the formation of a peptide bond.

As used herein, a “¹⁰Fn3 domain” or “¹⁰Fn3 moiety” or “¹⁰Fn3 molecule” refers to wild-type ¹⁰Fn3 and biologically active variants thereof, e.g., biologically active variants that specifically bind to a target, such as a target protein. A wild-type human ¹⁰Fn3 domain may comprise one of the amino acid sequences set forth in SEQ ID NO: 1-8. Biologically active variants of a wild-type human ¹⁰Fn3 domain include ¹⁰Fn3 domains that comprise at least, at most or about 1, 2, 3, 4, 5, 6, 7, 8, 9, 10, 11, 12, 13, 14, 15, 16, 17, 18, 19, 20, 21, 22, 23, 24, 25, 30, 35, 40 or 45 amino acid changes, i.e., substitutions, additions or deletions, relative to a ¹⁰Fn3 domain comprising any one of SEQ ID NOs: 1-8. A biologically active variant of a wild-type ¹⁰Fn3 domain may also comprise, or comprise at most, 1-3, 1-5, 1-10, 1-15, 1-10, 1-25, 1-30, 1-35, 1-40 or 1-45 amino acid changes relative to a ¹⁰Fn3 domain comprising any one of SEQ ID NOs: 1-8. In certain embodiments, a biologically active variant of a wild-type ¹⁰Fn3 domain does not comprise more than 1, 2, 3, 4, 5, 6, 7, 8, 9, 10, 11, 12, 13, 14, 15, 16, 17, 18, 19, 20, 21, 22, 23, 24, 25, 30, 35, 40 or 45 amino acid changes, i.e., substitutions, additions or deletions, relative to an ¹⁰Fn3 domain comprising any one of SEQ ID NOs: 1-8. Amino acid changes may be in a loop region, in a strand or in the N-terminal or C-terminal region. Exemplary degenerate ¹⁰Fn3 amino acid sequences allowing for amino acid changes in the loop regions are provided herein as SEQ ID NOs: 9-16.

By “polypeptide” is meant any sequence of two or more amino acids, regardless of length, post-translation modification, or function. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D).

A “region” of a ¹⁰Fn3 domain (or moiety or molecule) as used herein refers to either a loop (AB, BC, CD, DE, EF and FG), a β-strand (A, B, C, D, E, F and G), the N-terminus (corresponding to amino acid residues 1-7 of SEQ ID NO: 1), or the C-terminus (corresponding to amino acid residues 93-94 of SEQ ID NO: 1).

A “north pole loop” of a ¹⁰Fn3 domain (or moiety) refers to any one of the BC, DE and FG loops of a ¹⁰Fn3 domain.

A “south pole loop” of a ¹⁰Fn3 domain (or moiety) refers to any one of the AB, CD and EF loops of a ¹⁰Fn3 domain.

A “scaffold region” refers to any non-loop region of a human ¹⁰Fn3 domain. The scaffold region includes the A, B, C, D, E, F and G β-strands as well as the N-terminal region (amino acids corresponding to residues 1-7 of SEQ ID NO: 1) and the C-terminal region (amino acids corresponding to residues 93-94 of SEQ ID NO: 1).

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST℠, BLAST℠-2, ALIGN, ALIGN-2 or Megalign (DNASTAR®) software. Those skilled in the art can determine appropriate parameters for measuring alignment, including any algorithms needed to achieve maximal alignment over the full-length of the sequences being compared.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by a sequence alignment program, such as BLAST℠, BLAST℠_2, ALIGN, ALIGN-2 or Megalign (DNASTAR®), in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

As used herein, an amino acid residue in a polypeptide is considered to “contribute to binding” a target if (1) any of the non-hydrogen atoms of the residue's side chain or main chain is found to be within five angstroms of any atom of the binding target based on an experimentally determined three-dimensional structure of the complex, and/or (2) mutation of the residue to its equivalent in wild-type ¹⁰Fn3 (e.g., SEQ ID NO: 1), to alanine, or to a residue having a similarly sized or smaller side chain than the residue in question, leads to a measured increase of the equilibrium dissociation constant to the target (e.g., an increase in the kₒₙ).

The serum or plasma “half-life” of a polypeptide can generally be defined as the time taken for the serum concentration of the polypeptide to be reduced by 50%, in vivo, for example due to degradation of the polypeptide and/or clearance or sequestration of the polypeptide by natural mechanisms. The half-life can be determined in any manner known per se, such as by pharmacokinetic analysis. Suitable techniques will be clear to the person skilled in the art, and may, for example, generally involve the steps of administering a suitable dose of a polypeptide to a primate; collecting blood samples or other samples from said primate at regular intervals; determining the level or concentration of the polypeptide in said blood sample; and calculating, from (a plot of) the data thus obtained, the time until the level or concentration of the polypeptide has been reduced by 50% compared to the initial level upon dosing. Methods for determining half-life may be found, for example, in Kenneth et al., Chemical Stability of Pharmaceuticals: A Handbook for Pharmacists (1986); Peters et al., Pharmacokinete Analysis: A Practical Approach (1996); and Gibaldi, M. et al., Pharmacokinetics, Second Rev. Edition, Marcel Dekker (1982).

Serum half-life can be expressed using parameters such as the t½-alpha, t½-beta and the area under the curve (AUC). An “increase in half-life” refers to an increase in any one of these parameters, any two of these parameters, or in all three these parameters. In certain embodiments, an increase in half-life refers to an increase in the t½-beta, either with or without an increase in the t½-alpha and/or the AUC or both.

“Shelf-life” of a pharmaceutical product, e.g., a protein comprising an FBS moiety and an HSA moiety, is the length of time the product is stored before decomposition occurs. For example, shelf-life may be defined as the time for decomposition of 0.1%, 0.5%, 1%, 5%, or 10% of the product.

### Overview

Provided herein are proteins comprising a fibronectin based scaffold (FBS) domain, e.g., Fn3, such as ¹⁰Fn3 molecules, that bind specifically to a target, and wherein the FBS domain is linked at its C-terminus to a region consisting of PmXn, wherein P is proline, X is any amino acid and wherein n is 0 or an integer that is at least 1 and m is an integer that is at least 1. The application is based at least in part on the discovery that adding a proline and optionally one or more amino acids at the C-terminus of a ¹⁰Fn3 molecule increases at least one characteristic of the ¹⁰Fn3 molecule, e.g., its thermostability or solubility, relative to the unmodified ¹⁰Fn3 molecule.

The ¹⁰Fn3 molecules described herein may be designed to bind to any target of interest. In exemplary embodiments, the target is an antigen, a polypeptide or a therapeutic protein target of interest. Exemplary therapeutically desirable targets, include, for example, tumor necrosis factor alpha (TNF-alpha), VEGFR2, PCSK9, IL-23, EGFR and IGF1R.

### Fibronectin Based Scaffolds

As used herein, a “fibronectin based scaffold” or “FBS” protein or moiety refers to proteins or moieties that are based on a fibronectin type III (“Fn3”) repeat. Fn3 is a small (about 10 kDa) domain that has the structure of an immunoglobulin (Ig) fold (i.e., an Ig-like β-sandwich structure, consisting of seven β-strands and six loops). Fibronectin has 18 Fn3 repeats, and while the sequence homology between the repeats is low, they all share a high similarity in tertiary structure. Fn3 domains are also present in many proteins other than fibronectin, such as adhesion molecules, cell surface molecules, e.g., cytokine receptors, and carbohydrate binding domains. For reviews see Bork et al., Proc. Natl. Acad. Sci. USA, 89 (19): 8990-8994 (1992); Bork et al., J. Mol. Biol., 242 (4): 309-320 (1994); Campbell et al., Structure, 2 (5): 333-337 (1994); Harpez et al., J. Mol. Biol., 238 (4): 528-539 (1994)). The term “FBS” protein or moiety is intended to include scaffolds based on Fn3 domains from these other proteins (i.e., non fibronectin molecules).

An Fn3 domain is small, monomeric, soluble, and stable. It lacks disulfide bonds and, therefore, is stable under reducing conditions. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel β-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”).

The loops in Fn3 molecules are structurally similar to complementary determining regions (CDRs) of antibodies, and when altered, may be involved in binding of the Fn3 molecule to a target, e.g., a target protein. Other regions of Fn3 molecules, such as the beta or beta-like strands and N-terminal or C-terminal regions, when altered, may also be involved in binding to a target. Any or all of loops AB, BC, CD, DE, EF and FG may participate in binding to a target. Any of the beta or beta-like strands may be involved in binding to a target. Fn3 domains may also bind to a target through one or more loops and one or more beta or beta-like strands. Binding may also require the N-terminal or C-terminal regions. An FBS domain for use in a protein may comprise all loops, all beta or beta-like strands, or only a portion of them, wherein certain loops and/or beta or beta-like strands and/or N- or C-terminal regions are modified (or altered), provided that the FBS domain preferably binds specifically to a target. For example, an FBS domain may comprise 1, 2, 3, 4, 5 or 6 loops, 1, 2, 3, 4, 5, 6, 7, or 8 beta strands, and optionally an N-terminal and/or C-terminal region, wherein one or more loops, one or more beta strands, the N-terminal region and/or the C-terminal regions are modified relative to the wild-type FBS domain.

In exemplary embodiments, ligand (or target) binding FBS moieties described herein are based on the tenth fibronectin type III domain, i.e., the tenth module of Fn3 (¹⁰Fn3). The amino acid sequence of a wild-type human ¹⁰Fn3 moiety is as follows:

(SEQ ID NO: 1)
VSDVPRDLEVVAATPTSLLISWDAPAVTVRYYRITYGETGGNSPVQE
FTVPGSKSTATISGLKPGVDYTITVYAVTGRGDSPASSKPISINYRT

(the AB, CD and EF loops are underlined; the BC, FG, and DE loops are emphasized in bold; the β-strands are located between or adjacent to each of the loop regions; and the N-terminal region is shown in italics). The last two amino acid residues of SEQ ID NO: 1 are a portion of a C-terminal region.

Wild-type human ¹⁰Fn3 molecules also include those lacking the N-terminal region or a portion thereof. For example, a wild-type ¹⁰Fn3 molecule may comprise SEQ ID NO: 1, wherein amino acid residues 1, 1-2, 1-3, 1-4, 1-5, 1-6 or 1-7 are deleted (SEQ ID NOs: 2-8, respectively). Table 1 shows the amino acid sequence of these wild-type human ¹⁰Fn3 moieties:

TABLE 1
Amino acid sequences of wild-type human ¹⁰Fn3 molecules
with various N-terminal regions

| | N-terminal | | |
|---|---|---|---|
| Version | region | Wild-type human ¹⁰Fn3 core domain | Full length |
| 1 | VSDVPRD | LEVVAATPTSLLISWDAPAVTVRYYRITY | SEQ ID NO: 1 |
| | (SEQ ID NO: | GETGGNSPVQEFTVPGSKSTATISGLKPG | |
| | 19) | VDYTITVYAVTGRGDSPASSKPISINYRT | |
| | | (SEQ ID NO: 17) | |
| 2 | SDVPRD | LEVVAATPTSLLISWDAPAVTVRYYRITY | SEQ ID NO: 2 |
| | (SEQ ID NO: | GETGGNSPVQEFTVPGSKSTATISGLKPG | |
| | 20) | VDYTITVYAVTGRGDSPASSKPISINYRT | |
| | | (SEQ ID NO: 17) | |
| 3 | DVPRD | LEVVAATPTSLLISWDAPAVTVRYYRITY | SEQ ID NO: 3 |
| | (SEQ ID NO: | GETGGNSPVQEFTVPGSKSTATISGLKPG | |
| | 21) | VDYTITVYAVTGRGDSPASSKPISINYRT | |
| | | (SEQ ID NO: 17) | |
| 4 | VPRD | LEVVAATPTSLLISWDAPAVTVRYYRITY | SEQ ID NO: 4 |
| | (SEQ ID NO: | GETGGNSPVQEFTVPGSKSTATISGLKPG | |
| | 22) | VDYTITVYAVTGRGDSPASSKPISINYRT | |
| | | (SEQ ID NO: 17) | |
| 5 | PRD | LEVVAATPTSLLISWDAPAVTVRYYRITY | SEQ ID NO: 5 |
| | | GETGGNSPVQEFTVPGSKSTATISGLKPG | |
| | | VDYTITVYAVTGRGDSPASSKPISINYRT | |
| | | (SEQ ID NO: 17) | |
| 6 | RD | LEVVAATPTSLLISWDAPAVTVRYYRITY | SEQ ID NO: 6 |
| | | GETGGNSPVQEFTVPGSKSTATISGLKPG | |
| | | VDYTITVYAVTGRGDSPASSKPISINYRT | |
| | | (SEQ ID NO: 17) | |
| 7 | D | LEVVAATPTSLLISWDAPAVTVRYYRITY | SEQ ID NO: 7 |
| | | GETGGNSPVQEFTVPGSKSTATISGLKPG | |
| | | VDYTITVYAVTGRGDSPASSKPISINYRT | |
| | | (SEQ ID NO: 17) | |
| 8 | — | LEVVAATPTSLLISWDAPAVTVRYYRITY | SEQ ID NO: 8 |
| | | GETGGNSPVQEFTVPGSKSTATISGLKPG | |
| | | VDYTITVYAVTGRGDSPASSKPISINYRT | |
| | | (SEQ ID NO: 17) | |

In some embodiments, the AB loop corresponds to residues 14-17, the BC loop corresponds to residues 23-31, the CD loop corresponds to residues 37-47, the DE loop corresponds to residues 51-56, the EF loop corresponds to residues 63-67, and the FG loop corresponds to residues 75-87 of SEQ ID NO: 1. The BC, DE and FG loops align along one face of the molecule, i.e., the “north pole”, and the AB, CD and EF loops align along the opposite face of the molecule, i.e., the “south pole”. In SEQ ID NO: 1, β-strand A corresponds to residues 8-13, β-strand B corresponds to residues 18-22, β-strand C corresponds to residues 32-36, beta strand D corresponds to residues 48-50, β-strand E corresponds to residues 57-62, β-strand F corresponds to residues 68-74, and β-strand G corresponds to residues 88-92. The β-strands are connected to each other through the corresponding loop, e.g., strands A and B are connected via loop AB in the formation β-strand A, loop AB, β-strand B, etc.

An example of FBS proteins that are based on human ¹⁰Fn3 domains are adnectins (Adnexus, a wholly owned subsidiary of Bristol-Myers Squibb). Adnectins are ¹⁰Fn3 molecules in which CDR-like loop regions, β-strands, N-terminal and/or C-terminal regions of a ¹⁰Fn3 domain has been modified to evolve a protein capable of binding to a compound of interest. For example, U.S. Pat. No. 7,115,396 describes ¹⁰Fn3 domain proteins wherein alterations to the BC, DE, and FG loops result in high affinity TNFα binders. U.S. Pat. No. 7,858,739 describes Fn3 domain proteins wherein alterations to the BC, DE, and FG loops result in high affinity VEGFR2 binders.

In certain embodiments, the FBS moiety comprises a ¹⁰Fn3 domain that is defined generally by the following degenerate sequence:

(SEQ ID NO: 9)
VSDVPRDLEVVAA(X)ᵤLLISW(X)ᵥYRITY(X)_{w}FTV(X)ₓATISGL
(X)_{y}YTITVYA(X)_{z}ISINYRT,

or by a sequence selected from the group of SEQ ID NO: 10-16, which sequences are identical to SEQ ID NO: 9, except that they are lacking 1, 2, 3, 4, 5, 6 or 7 N-terminal amino acids, respectively. Table 2 shows the amino acid sequences of these degenerate human ¹⁰Fn3 molecules.

TABLE 2
Amino acid sequences of degenerate wild-type human ¹⁰Fn3
molecules with various N-terminal regions

| | N-terminal | Degenerate wild-type | |
|---|---|---|---|
| Version | region | human ¹⁰Fn3 core domain | Full length |
| 1 | VSDVPRD | LEVVAA(X)ᵤLLISW(X)ᵥYRITY(X)_{w}FTV | SEQ ID NO: 9 |
| | (SEQ ID NO: | (X)ₓATISGL(X)_{y}YTITVYA(X)_{z}ISINYRT | |
| | 19) | (SEQ ID NO: 18) | |
| 2 | SDVPRD | LEVVAA(X)ᵤLLISW(X)ᵥYRITY(X)_{w}FTV | SEQ ID NO: 10 |
| | (SEQ ID NO: | (X)ₓATISGL(X)_{y}YTITVYA(X)_{z}ISINYRT | |
| | 20) | (SEQ ID NO: 18) | |
| 3 | DVPRD | LEVVAA(X)ᵤLLISW(X)ᵥYRITY(X)_{w}FTV | SEQ ID NO: 11 |
| | (SEQ ID NO: | (X)ₓATISGL(X)_{y}YTITVYA(X)_{z}ISINYRT | |
| | 21) | (SEQ ID NO: 18) | |
| 4 | VPRD | LEVVAA(X)ᵤLLISW(X)ᵥYRITY(X)_{w}FTV | SEQ ID NO: 12 |
| | (SEQ ID NO: | (X)ₓATISGL(X)_{y}YTITVYA(X)_{z}ISINYRT | |
| | 22) | (SEQ ID NO: 18) | |
| 5 | PRD | LEVVAA(X)ᵤLLISW(X)ᵥYRITY(X)_{w}FTV | SEQ ID NO: 13 |
| | | (X)ₓATISGL(X)_{y}YTITVYA(X)_{z}ISINYRT | |
| | | (SEQ ID NO: 18) | |
| 6 | RD | LEVVAA(X)ᵤLLISW(X)ᵥYRITY(X)_{w}FTV | SEQ ID NO: 14 |
| | | (X)ₓATISGL(X)_{y}YTITVYA(X)_{z}ISINYRT | |
| | | (SEQ ID NO: 18) | |
| 7 | D | LEVVAA(X)ᵤLLISW(X)ᵥYRITY(X)_{w}FTV | SEQ ID NO: 15 |
| | | (X)ₓATISGL(X)_{y}YTITVYA(X)_{z}ISINYRT | |
| | | (SEQ ID NO: 18) | |
| 8 | — | LEVVAA(X)ᵤLLISW(X)ᵥYRITY(X)_{w}FTV | SEQ ID NO: 16 |
| | | (X)ₓATISGL(X)_{y}YTITVYA(X)_{z}ISINYRT | |
| | | (SEQ ID NO: 18) | |

In SEQ ID NOs: 25-32 and 50, the AB loop is represented by (X)ᵤ, the BC loop is represented by (X)ᵥ, the CD loop is represented by (X)_{w}, the DE loop is represented by (X)ₓ, the EF loop is represented by (X)_{y} and the FG loop is represented by X_{z}. X represents any amino acid and the subscript following the X represents an integer of the number of amino acids. In particular, u, v, w, x, y and z may each independently be anywhere from 2-20, 2-15, 2-10, 2-8, 5-20, 5-15, 5-10, 5-8, 6-20, 6-15, 6-10, 6-8, 2-7, 5-7, or 6-7 amino acids. The sequences of the beta strands (underlined in SEQ ID NO: 9) may have anywhere from 0 to 10, from 0 to 8, from 0 to 6, from 0 to 5, from 0 to 4, from 0 to 3, from 0 to 2, or from 0 to 1 substitutions, deletions or additions across all 7 scaffold regions relative to the corresponding amino acids shown in SEQ ID NOs: 9-16. In some embodiments, the sequences of the beta strands may have anywhere from 0 to 10, from 0 to 8, from 0 to 6, from 0 to 5, from 0 to 4, from 0 to 3, from 0 to 2, or from 0 to 1 substitutions, e.g., conservative substitutions, across all 7 scaffold regions relative to the corresponding amino acids shown in SEQ ID NO: 9-16.

In certain embodiments, the hydrophobic core amino acid residues (bolded residues in SEQ ID NO: 9 above) are fixed, and any substitutions, conservative substitutions, deletions or additions occur at residues other than the hydrophobic core amino acid residues. Thus, in some embodiments, the hydrophobic core residues of the polypeptides provided herein have not been modified relative to the wild-type human ¹⁰Fn3 domain (e.g., SEQ ID NO: 1).

In some embodiments, an FBS moiety comprises a ¹⁰Fn3 domain, wherein the ¹⁰Fn3 domain comprises a loop, AB; a loop, BC; a loop, CD; a loop, DE; a loop, EF; and a loop, FG; and has at least one loop selected from loop AB, BC, CD, DE, EF and FG with an altered amino acid sequence relative to the sequence of the corresponding loop of the wild-type human ¹⁰Fn3 domain. In some embodiments, a single loop is altered. In some embodiments, at most 2 loops are altered. In some embodiments, at most 3 loops are altered. In some embodiments, the BC, DE and/or FG loops are altered. In certain embodiments, the AB, CD and EF loops are altered. In certain embodiments, the FG loop is the only loop that is altered. In certain embodiments, the CD loop is the only loop that is altered. In other embodiments, the CD and FG loops are both altered, and optionally, no other loops are altered. In certain embodiments, the CD and EF loops are both altered, and optionally, no other loops are altered. In some embodiments, one or more specific scaffold alterations are combined with one or more loop alterations. By “altered” is meant one or more amino acid sequence alterations relative to a template sequence (i.e., the corresponding wild-type human fibronectin domain) and includes amino acid additions, deletions, and substitutions. Exemplary ¹⁰Fn3 molecules comprising specific combinations of altered loops and/or scaffold regions (e.g., beta strands, N-terminal region and C-terminal region) are further disclosed herein.

It should be understood that not every residue within a loop region needs to be modified in order to achieve a ¹⁰Fn3 binding domain having strong affinity for a desired target. Additionally, insertions and deletions in the loop regions may also be made while still producing high affinity ¹⁰Fn3 binding domains.

In some embodiments, one or more loops selected from AB, BC, CD, DE, EF and FG may be extended or shortened in length relative to the corresponding loop in wild-type human ¹⁰Fn3. In any given polypeptide, one or more loops may be extended in length, one or more loops may be reduced in length, or combinations thereof. In some embodiments, the length of a given loop may be extended by 2-25, 2-20, 2-15, 2-10, 2-5, 5-25, 5-20, 5-15, 5-10, 10-25, 10-20, or 10-15 amino acids. In some embodiments, the length of a given loop may be reduced by 1-15, 1-11, 1-10, 1-5, 1-3, 1-2, 2-10, or 2-5 amino acids. In particular, the FG loop of ¹⁰Fn3 is 13 residues long, whereas the corresponding loop in antibody heavy chains ranges from 4-28 residues. To optimize antigen binding in polypeptides relying on the FG for target binding, therefore, the length of the FG loop of ¹⁰Fn3 may be altered in length as well as in sequence to obtain the greatest possible flexibility and affinity in target binding.

In some embodiments, the FBS moiety comprises a ¹⁰Fn3 domain wherein the non loop regions comprise an amino acid sequence that is at least 80, 85, 90, 95, 98, or 100% identical to the non-loop regions of SEQ ID NO: 1, wherein at least one loop selected from AB, BC, CD, DE, EF and FG is altered. For example, in certain embodiments, the

AB loop may have up to 4 amino acid substitutions, up to 10 amino acid insertions, up to 3 amino acid deletions, or a combination thereof; the BC loop may have up to 10 amino acid substitutions, up to 4 amino acid deletions, up to 10 amino acid insertions, or a combination thereof; the CD loop may have up to 6 amino acid substitutions, up to 10 amino acid insertions, up to 4 amino acid deletions, or a combination thereof; the DE loop may have up to 6 amino acid substitutions, up to 4 amino acid deletions, up to 13 amino acid insertions, or a combination thereof; the EF loop may have up to 5 amino acid substitutions, up to 10 amino acid insertions, up to 3 amino acid deletions, or a combination thereof; and/or the FG loop may have up to 12 amino acid substitutions, up to 11 amino acid deletions, up to 25 amino acid insertions, or a combination thereof.

In some embodiments, an FBS moiety comprises a ¹⁰Fn3 domain having at least 40%, 50%, 55%, 60%, 65%, 70%, 75%, 80%, 85%, or 90% identity to a human ¹⁰Fn3 domain having an amino acid sequence selected from the group of sequence comprising SEQ ID NOs: 1-16. In certain embodiments, the FBS moiety provided herein has at least 50% identity to an amino acid sequence selected from the group of amino acid sequences comprising SEQ ID NO: 1-16. In other embodiments, the FBS moiety has at least 65% identity to an amino acid sequence selected from the group of amino acid sequences comprising SEQ ID NO: 1-16. In certain embodiments, one or more of the loops will not be modified relative to the sequence of the corresponding loop of the wild-type sequence and/or one or more of the β-strands will not be modified relative to the sequence of the corresponding β-strand of the wild-type sequence and/or the N-terminal or C-terminal regions will not be modified. In certain embodiments, each of the beta or beta-like strands of a ¹⁰Fn3 domain in an FBS moiety may comprise, consist essentially of, or consist of an amino acid sequence that is at least 80%, 85%, 90%, 95% or 100% identical to the sequence of a corresponding beta or beta-like strand of SEQ ID NO: 1. Preferably, variations in the β-strand regions will not disrupt the stability of the polypeptide in physiological conditions.

In some embodiments, the non-loop region of a ¹⁰Fn3 domain may be modified by one or more conservative substitutions. As many as 5%, 10%, 20% or even 30% or more of the amino acids in the ¹⁰Fn3, domain may be altered by a conservative substitution without substantially altering the affinity of the ¹⁰Fn3 for a ligand. In certain embodiments, the non-loop regions, e.g., the β-strands may comprise anywhere from 0-15, 0-10, 0-8, 0-6, 0-5, 0-4, 0-3, 1-15, 1-10, 1-8, 1-6, 1-5, 1-4, 1-3, 2-15, 2-10, 2-8, 2-6, 2-5, 2-4, 5-15, or 5-10 conservative amino acid substitutions. In exemplary embodiments, the scaffold modification may reduce the binding affinity of the ¹⁰Fn3 binder for a ligand by less than 100-fold, 50-fold, 25-fold, 10-fold, 5-fold, or 2-fold. It may be that such changes may alter the immunogenicity of the ¹⁰Fn3 in vivo, and where the immunogenicity is decreased, such changes may be desirable. As used herein, “conservative substitutions” are residues that are physically or functionally similar to the corresponding reference residues. That is, a conservative substitution and its reference residue have similar size, shape, electric charge, chemical properties including the ability to form covalent or hydrogen bonds, or the like. Exemplary conservative substitutions include those fulfilling the criteria defined for an accepted point mutation in Dayhoff et al., Atlas of Protein Sequence and Structure, 5:345-352 (1978 and Supp.). Examples of conservative substitutions include substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

Also provided herein are ¹⁰Fn3 domains having combinations of loop and scaffold modifications. Conjugates may comprise a ¹⁰Fn3, domain comprising (i) a modification in the amino acid sequence of at least one of loops AB, BC, CD, DE, EF, or FG, and (ii) a modification in the amino acid sequence of at least one scaffold region (i.e., a modification in at least one β-strand, the N-terminal region, and/or the C-terminal region), wherein the modified loop(s) and modified scaffold region(s) both contribute to binding the same target. In exemplary embodiments, the scaffold region modifications are located adjacent to modifications in a loop region, e.g., if the AB loop is modified, scaffold mutations may tend to be located in β-strand A and/or β-strand B, which are adjacent to the AB loop in the linear sequence of the ¹⁰Fn3 domain. In other embodiments, a cluster of modifications may be found together in loop and scaffold regions that are adjacent to one another in the linear sequence of the Fn3 domain. For example, Fn3 binders having both loop and scaffold modifications, may have clusters of amino acid modifications in the following combinations of loop and scaffold regions that are adjacent to each other in the linear sequence of the Fn3 domain: β-strand/loop/β-strand, loop/β-strand/loop, loop/β-strand/loop/β-strand, terminal region/β-strand/loop, or loop/β-strand/terminal region, etc. For example, Fn3 domains having novel combinations of loop and scaffold modifications may have clusters of modifications such that over a stretch of 20 contiguous amino acids at least 15 of the amino acids are modified relative to wild-type. In other embodiments, at least 17 out of 20, 18 out of 20, 17 out of 25, 20 out of 25, or 25 out of 30 residues in a contiguous stretch are modified relative to the wild-type Fn3 domain sequence over the corresponding stretch of amino acids. In certain embodiments, a given Fn3 domain may have two or three clusters of modifications separated by stretches of unmodified (i.e., wild-type) sequence. For any given region (i.e., a loop, β-strand or terminal region) that is modified, all or only a portion of the region may be modified relative to the wild-type sequence. When a β-strand region is modified, preferably the hydrophobic core residues remain unmodified (i.e., wild-type) and one or more of the non-core residues in the β-strand are modified.

In some embodiments, ¹⁰Fn3 domains comprise a binding face along the “west-side” of the molecule (“West-side binders” or “WS binders”). WS binders may comprise a modified CD loop and a modified FG loop, as compared to the corresponding CD and FG loop sequences set forth in SEQ ID NO: 1. The CD loop and the FG loop both contribute to binding to the same target. In certain embodiments, the WS binders may comprise additional modifications at one or more regions within the Fn3 domain. For example, WS binders may comprise scaffold modifications in one or more of the β-strand regions adjacent to the CD and/or FG loops. In particular, WS binders may comprise sequence modifications in one or more of β-strand C, β-strand D, β-strand F, and/or β-strand G. Exemplary scaffold modifications include modifications at one or more scaffold region positions corresponding to the amino acid positions: 33, 35, 49, 69, 71, 73, 89 and/or 91 of SEQ ID NO: 1. The WS binders may also comprise modifications in the BC loop, particularly in the C-terminal portion of the BC loop. In one embodiment, the last two residues of the BC loop (i.e., corresponding to amino acids 30 and 31 in the wild-type ¹⁰Fn3 domain) are modified relative to the wild-type sequence. All or a portion of the additional loop and scaffold modifications may contribute to binding to the target in conjunction with the modified CD and FG loops. Preferably, the hydrophobic core residues are not modified relative to the wild-type sequence.

Exemplary WS binders include those having a wild-type or mutated amino acid at positions 30, 31, 33, 35, 37, 38, 46, 47, 49, 50, 67, 69, 71, 73, 75, 76, 84, 85, 86, 87, 89 or 91.

In some embodiments, a ¹⁰Fn3 domain comprises modifications in the CD, DE and, in some cases, EF loops, wherein the loop modifications all contribute to target binding. These polypeptides are referred to as “front binders”. The front binders may additionally comprise modifications in one or more scaffold regions, particularly in scaffold regions that flank or are adjacent to a modified loop region. For example, the front binders may comprise a scaffold modification in one or more of β-strand C, β-strand D, and/or β-strand E relative to the sequences of the corresponding β-strands of the wild-type Fn3 domain, e.g., human ¹⁰Fn3 domain (SEQ ID NO: 1). Preferably the hydrophobic core residues are not modified relative to the wild-type sequence. Exemplary scaffold modifications that may be present in front binders, include modifications at one or more positions corresponding to amino acid positions 36, 49, 58 and/or 50 of SEQ ID NO: 1. Such scaffold modifications may contribute to binding to the target together with the modified loops. In certain embodiments, the front binders may comprise clusters of modifications spanning several loop and strand regions of the Fn3, e.g., ¹⁰Fn3, domain. In particular, the front binders may comprise modifications in at least 15, 20, 24, 25, or 27 of the 31 residues between the amino acids corresponding to residues 36 through 66 of the wild-type Fn3, e.g., human ¹⁰Fn3, domain (SEQ ID NO: 1). The loop and/or strand modifications may include amino acid substitutions, deletions and/or insertions, or combinations thereof. In exemplary embodiments, the CD loop is extended in length or reduced in length relative to the CD loop of the Fn3, e.g., wild-type human ¹⁰Fn3, domain (SEQ ID NO: 1).

In some embodiments, ¹⁰Fn3 domains comprise modifications in the EF and FG loops, wherein the loop modifications contribute to binding the same target. These polypeptides are referred to as “back binders” herein. The back binders may comprise additional modifications in other loop and/or scaffold regions. For example, a back binder may contain modifications in at least a portion of the AB loop, preferably the N-terminal portion of the AB loop. In an exemplary embodiment, the first two amino acids of the AB loop (i.e., corresponding to amino acid residues 14 and 15 of the wild-type ¹⁰Fn3 domain) are modified relative to the wild-type sequence. In certain embodiments, a back binder may also contain one or more scaffold modifications, particularly modifications in one or more scaffold regions that are adjacent to a modified loop region. For example, back binders may contain one or more modifications in one or more of β-strand A, β-strand G, the N-terminal region, and/or the C-terminal region. Preferably the hydrophobic core residues are not modified relative to the wild-type sequence. Exemplary scaffold modifications include modifications at one or more positions corresponding to amino acid positions 1-7, 9-13, 89, 91, 93 and/or 94 of SEQ ID NO: 1. One or more of the additional loop and/or scaffold modifications may contribute to binding to the target along with the modified EF and FG loops. Suitable loop and/or scaffold region modifications include amino acid substitutions, deletions and/or insertions, or combinations thereof. In certain embodiments, the amino acid sequence of the FG loop is extended in length or reduced in length relative to the FG loop of the wild-type human ¹⁰Fn3 domain (SEQ ID NO: 1).

In certain embodiments, a back binder may comprise a cluster of modified amino acid residues over a contiguous span of several regions in the ¹⁰Fn3 domain. For example, at least 14 of the first 15 amino acid residues of the Fn3, e.g., ¹⁰Fn3, domain may be modified relative to the corresponding residues in the wild-type Fn3, e.g., human ¹⁰Fn3, domain (SEQ ID NO: 1), and/or at least 15 of the 18 residues between the amino acids corresponding to residues 80 through 97 (or 94) of the wild-type Fn3, e.g., human ¹⁰Fn3, domain (SEQ ID NO: 1 or 23) may be modified relative to the corresponding residues in the wild-type sequence. When referring to amino acids at positions further C-terminal to 94 in a ¹⁰Fn3 molecule, it is in the context of a ¹⁰Fn3 molecule that comprises the flexible linker between the 10^{th} and 11^{th} repeat of the Fn3 domain, i.e., EIDKPSQ (SEQ ID NO: 113), thus forming a 101 amino acid long protein. Thus, SEQ ID NO: 1 linked to EIDKPSQ (SEQ ID NO: 113) at its C-terminus is represented by SEQ ID NO: 23.

(SEQ ID NO: 23)
VSDVPRDLEVVAATPTSLLISWDAPAVTVRYYRITYGETGGNSPVQEFTV
PGSKSTATISGLKPGVDYTITVYAVTGRGDSPASSKPISINYRT EIDKP
SQ

In certain embodiments, a ¹⁰Fn3 domain comprises modifications in the amino acid sequences of β-strand A, loop AB, β-strand B, loop CD, β-strand E, loop EF, and β-strand F, relative to the sequences of the corresponding regions of the wild-type sequence. These polypeptides are referred to as “south pole binders” or “SP binders” herein. The modified loops and strands contribute to binding to the same target. The amino acid sequence of the CD loop may be extended in length or reduced in length relative to the CD loop of the wild-type Fn3, e.g., human ¹⁰Fn3, domain (SEQ ID NO: 1 or 23). The south pole binders may comprise additional modifications in-strand G and/or the C-terminal region relative to the sequence of the corresponding region of the wild-type sequence. In exemplary embodiments, the south pole binders may comprise one or more modifications at amino acids corresponding to positions 11, 12, 19, 60, 61, 69, 91, 93 and 95-97 of the wild-type sequence.

In some embodiments, a ¹⁰Fn3 domain comprises modified BC, DE and FG loops, as compared to the corresponding BC, DE and FG loop sequences set forth in SEQ ID NO: 1 or 23, as well as additional modifications in one or more of β-strand C, β-strand D, β-strand F and β-strand G strand residues. The β-strand and loop region modifications together contribute to binding to the target. These proteins are referred to as “Northwest binders”, or “NW binders”, herein. In exemplary embodiments, the NW binders comprise one or more scaffold modifications at any one of, or combination of, amino acid positions corresponding to scaffold region positions R33, T49, Y73 and S89 of SEQ ID NO: 1 or 23. Suitable modifications in loop and scaffold regions include amino acid substitutions, deletions and/or insertions, or combinations thereof. In certain embodiments, one or more of the BC, DE and FG loops are extended in length or reduced in length, or combinations thereof, relative to the wild-type sequence. In one embodiment, each of the BC, DE and FG loops are extended in length or reduced in length, or combinations thereof, relative to the wild-type sequence (e.g., SEQ ID NO: 1 or 23). In certain embodiments, only a portion of the BC loop is modified, particularly the C-terminal portion, relative to the wild-type sequence. For example, the BC loop may be modified only at amino acid residues corresponding to amino acids 27-31 of the wild-type BC loop, whereas the rest of the BC loop (i.e., corresponding to residues 23-26 of the wild-type loop) are left unmodified.

In some embodiments, a ¹⁰Fn3 domain comprises a modified BC, DE and FG loop as well as one or more additional modifications in any one of, or combination of, the N-terminal region, β-strand A, β-strand B and/or β-strand E. These proteins are referred to as “Northeast binders”, or “NE binders”, herein. In exemplary embodiments, the NE binders are modified at any one of, or combination of, amino acids corresponding to scaffold region positions 1-7, E9, L19, S21 and/or T58 of the wild-type sequence (SEQ ID NO: 1 or 23). The combination of modified loop and scaffold regions contributes to binding to the target.

In some embodiments, a ¹⁰Fn3 domain comprises modifications in one or more of the AB, CD, DE and EF loops, as well as additional modifications in one or more of β-strand B, β-strand D and/or β-strand E. These proteins are referred to as “South Front binders” herein. The combination of modified loop and strand residues contributes to binding to the target. In exemplary embodiments, a South Front binder may be modified at one or more amino acid positions corresponding to scaffold region positions L19, T49, T58, S60, and/or G61 of SEQ ID NO: 1 or 23 and/or at one or more amino acid positions corresponding to loop region positions T14-S17, P51, T56, G40-E47, and/or K63-G65 of SEQ ID NO: 1 or 23. In exemplary embodiments, a South Front binder may be extended in length or reduced in length in the AB loop, between amino acids corresponding to residues 18 and 20 of the wild-type sequence, and/or in the CD loop.

In some embodiments, a ¹⁰Fn3 domain comprises a modified β-strand A and β-strand G, as compared to the corresponding strand of SEQ ID NO: 1 or 23. These proteins are referred to as “AG Binders” or “AG Strand” binders herein. In certain embodiments, the AG strand binders comprise clusters of modifications at the N-terminal and C-terminal portions of the Fn3, e.g., ¹⁰Fn3, domain, whereas the middle portion of the Fn3 remains unmodified. For example, an AG strand binder may comprise modifications at 16 out of 19 of the first 19 amino acids in the ¹⁰Fn3 domain (i.e., corresponding to amino acid positions 1-19 of SEQ ID NO: 1 or 23) and modifications at 13-17 out of 18 of the last 18 amino acids in the ¹⁰Fn3 domain (i.e., corresponding to amino acid positions 84-101 of SEQ ID NO: 9) or at 14-18 out of 22 of the last 22 amino acids in the ¹⁰Fn3 domain (i.e., corresponding to amino acid positions 80-101 of SEQ ID NO: 9). In exemplary embodiments, an AG binder may comprise modifications at one or more positions corresponding to positions 1-7, 9, 11-17, 19, 84-89 and 91-97 of SEQ ID NO: 9. Preferably the modified regions in an AG binder contribute to binding to the same target.

In some embodiments, a ¹⁰Fn3 domain comprises a modified CD and EF loop, as well as additional modifications in any one of, or combination of residues corresponding to positions 69 or 91-97 of SEQ ID NO: 1 or 23. These proteins are referred to as “Southwest binders”, or “SW binders”, herein. The modified loop and scaffold regions contribute to binding to the target.

In certain embodiments, proteins comprise a ¹⁰Fn3 domain having reduced immunogenicity, wherein a portion of the BC loop is left as wild-type. Preferably such polypeptides have lower immunogenicity relative to an equivalent polypeptide with modifications in a greater portion of the BC loop. In exemplary embodiments, the N-terminal portion of the BC loop is left as wild-type. For example, the first 1, 2, 3, 4, 5, or 5 residues of the BC loop may be left as wild-type, while the remaining C-terminal residues of the BC loop can be modified. In Fn3 designs having at least a portion of the N-terminal region of the BC loop as wild-type, it may be desirable to leave all or a portion of β-strand B and/or β-strand C unmodified relative to the wild-type sequence as well, particularly the portions of β-strand B and/or β-strand C that are adjacent to the BC loop (i.e., the C-terminal portion of β-strand B and/or the N-terminal portion of β-strand C). In exemplary embodiments, Fn3 domains having the wild-type sequence in an N-terminal portion of the BC loop and reduced immunogenicity may not have any modifications in the N-terminal region, β-strand A, AB loop, and β-strand B. In Fn3 designs with a portion of the BC loop as wild-type, the modified portion of the BC loop may contribute to target binding along with modifications in other regions of the ¹⁰Fn3 domain.

In certain embodiments, proteins comprise a ¹⁰Fn3 domain having reduced immunogenicity, wherein the strong HLA anchor in the region of β-strand B/BC loop/β-strand C (the “BC anchor”) has been removed or destroyed (e.g., modified relative to the wild-type sequence in a manner that reduces binding affinity to one or more HLA receptors). For example, the BC anchor may be removed or destroyed by modifying the Fn3, e.g., ¹⁰Fn3, domain at one or more positions corresponding to positions L19, S21, R33 and/or T35 of SEQ ID NO:1. When the BC anchor has been removed or destroyed, it is possible to modify the sequence of the BC loop without significantly increasing the immunogenic potential of the BC region. Accordingly, many such Fn3 designs have modifications in the BC loop in addition to the modifications in β-strand B and/or β-strand C. The BC loop may contribute to target binding, optionally in combination with modifications in other regions of the Fn3 domain. The modifications in β-strand B and/or β-strand C may or may not contribute to target binding.

In exemplary embodiments, an FBS, e.g., a ¹⁰Fn3 domain, binds to a desired target with a K_{d} of less than 500 nM, 100 nM, 50 nM, 10 nM, 5 nM, 1 nM, 500 μM, 100 pM or less. In some embodiments, the FBS, e.g., ¹⁰Fn3 domain, binds to a desired target with a K_{d} between 1 μM and 1 μM, between 100 μM and 500 nM, between 1 nM and 500 nM, or between 1 nM and 100 nM. In exemplary embodiments, the ¹⁰Fn3 moiety binds specifically to a target that is not bound by a wild-type ¹⁰Fn3 domain, particularly the wild-type human ¹⁰Fn3 domain having, e.g., SEQ ID NO: 1-8.

In certain embodiments, an FBS moiety comprises an amino acid sequence that is at least 40%, 50%, 60%, 70%, 75%, 80%, 85%, 90%, 95%, 97%, 98%, or 99% identical to an amino acid sequence selected from the group of sequences consisting of SEQ ID NOs: 1-16, and the FBS binds specifically to a target, e.g., with a K_{d} of less than 500 nM, 100 nM, 50 nM, 10 nM, 5 nM, 1 nM, 500 μM, 100 μM or less. The FBS moiety may comprise amino acid changes (or alterations) in one or more loops and one or more scaffold regions.

In some embodiments, one or more residues of the integrin-binding motif “arginine-glycine-aspartic acid” (RGD) (amino acids 78-80 of SEQ ID NO: 1) may be substituted so as to disrupt integrin binding. In some embodiments, the FG loop of the polypeptides provided herein does not contain an RGD integrin binding site. In one embodiment, the RGD sequence is replaced by a polar amino acid-neutral amino acid-acidic amino acid sequence (in the N-terminal to C-terminal direction). In certain embodiments, the RGD sequence is replaced with SGE or RGE.

In some embodiments, the amino acid sequences of the N-terminal and/or C-terminal regions of an FBS moiety are modified by deletion, substitution or insertion relative to the amino acid sequences of the corresponding regions of ¹⁰Fn3 domains comprising, e.g., SEQ ID NO: 1.

In certain embodiments, the amino acid sequence of the first 1, 2, 3, 4, 5, 6, 7, 8 or 9 residues of SEQ ID NO: 1 may be modified or deleted in the polypeptides provided herein relative to the sequence of the corresponding amino acids in the wild-type human ¹⁰Fn3 domain having SEQ ID NO: 1. In exemplary embodiments, the amino acids corresponding to amino acids 1-7, 8 or 9 of any one of SEQ ID NOs: 1-16 are replaced with an alternative N-terminal region having from 1-20, 1-15, 1-10, 1-8, 1-5, 1-4, 1-3, 1-2, or 1 amino acids in length. Exemplary alternative N-terminal regions include (represented by the single letter amino acid code) M, MG, G, MGVSDVPRDL (SEQ ID NO: 24) and GVSDVPRDL (SEQ ID NO: 25), or N-terminal truncations of any one of SEQ ID NOs: 24 or 25. Other suitable alternative N-terminal regions include, for example, XnSDVPRDL (SEQ ID NO: 26), XₙDVPRDL (SEQ ID NO: 27), Xₙ VPRDL (SEQ ID NO: 28), XₙPRDL (SEQ ID NO: 29), XₙRDL (SEQ ID NO: 30), XₙDL (SEQ ID NO: 31), or XₙL, wherein n=0, 1 or 2 amino acids, wherein when n=1, X is Met or Gly, and when n=2, X is Met-Gly. When a Met-Gly sequence is added to the N-terminus of a ¹⁰Fn3 domain, the M will usually be cleaved off, leaving a G at the N-terminus. In other embodiments, the alternative N-terminal region comprises the amino acid sequence MASTSG (SEQ ID NO: 32).

As further described herein, in some embodiments, the first seven or eight residues (i.e., residues 1-7 or 1-8) of SEQ ID NO: 1 are deleted, generating a ¹⁰Fn3 domain having the amino acid sequence of, e.g., SEQ ID NO: 8. Additional sequences may also be added to the N- or C-terminus of a ¹⁰Fn3 domain having the amino acid sequence of any one of SEQ ID NOs: 1-16. For example, in some embodiments, the N-terminal extension consists of an amino acid sequence selected from the group consisting of: M, MG, and G. For example, any one of SEQ ID NO: 1-16 may be preceded by M, MG, or G.

In certain embodiments, an FBS moiety is based on an Fn3 repeat other than the 10^{th} repeat of the type III domain of fibronectin, e.g., human fibronectin. For example, an FBS moiety may be similar to any of the other fibronectin type III repeats, e.g., the 1^{st}, 2^{nd}, 3^{rd}, 4^{th}, 5^{th}, 6^{th}, 7^{th}, 8^{th}, 9^{th}, 11^{th}, 12^{th}, 13^{th}, 14^{th}, 15^{th}, 16^{th}, 17^{th}, and 18^{th} Fn3 repeats. In yet other embodiments, an FBS moiety may be from a molecule other than fibronectin. Exemplary FBS moieties may be derived from tenascin, a protein that is composed of 15 Fn3 domains with similar sequence similarities to one another as found in fibronectin. These repeats are described, e.g., in Jacobs et al., Protein Engineering, Design & Selection, 25:107 (2012). Based on the homology of the repeats in the fibronectin molecule and those in the tenascin molecule, artificial molecules based on these homologies have been created. Proteins comprising a consensus amino acid sequence based on the homology of the domains in the fibronectin molecule are referred to as Fibcon and FibconB (WO 2010/093627 and Jacobs et al. (2012) supra.) and those based on the homology of the domains in the tenascin molecule are referred to as Tencon. An exemplary Fibcon amino acid sequence comprises the following amino acid sequence:

MPAPTDLRFTNETPSSLLISWTPPRVQITGYIIRYGPVGSDGRVKEFTV
PPSVSSATITGLKPGTEYTISVIALKDNQESEPLRGRVTTGG
(FibconB; SEQ ID NO: 33),

wherein loop AB consists of amino acids 13-16 (TPSS; SEQ ID NO: 34), loop BC consists of amino acids 22-28 (TPPRVQI; SEQ ID NO: 35), loop CD consists of amino acids 38-43 (VGSDGR; SEQ ID NO: 36), loop DE consists of amino acids 51-54 (PSVS; SEQ ID NO: 37), loop EF consists of amino acids 60-64 (GLKPG; SEQ ID NO: 38) and loop FG consist of amino acids 75-81 (KDNQESEP; SEQ ID NO:39). Another Fibcon amino acid sequence comprises the following amino acid sequence:

LDAPTDLQVTNVTDTSITVSWTPPSATITGYRITYTPSNGPGEPKELTV
PPSSTSVTITGITPGVEYVVSVYALKDNQESPPLVGTCTT (SEQ ID
NO: 40; Jacobs et al., supra).

Tenascin derived Fn3 proteins include Tencons (WO 2010/051274, WO 2010/051310 and WO 2011/137319, which are specifically incorporated by reference herein). An exemplary Tencon protein has the following amino acid sequence:

LPAPKNLVVSEVTEDSLRLSWTAPDAAFDSFLIQYQESEKVGEAINLTV
PGSERSYDLTGLKPGTEYTVSIYGVKGGHRSNPLSAEFTT (SEQ ID
NO: 41; Jacobs et al., supra, and WO 2011/137319),

wherein loop AB consists of amino acids 13-16 (TEDS; SEQ ID NO: 42, loop BC consists of amino acids 22-28 (TAPDAAF; SEQ ID NO: 43), loop CD consists of amino acids 38-43 (SEKVGE; SEQ ID NO: 44), loop DE consists of amino acids 51-54 (GSER; SEQ ID NO: 45), loop EF consists of amino acids 60-64 (GLKPG; SEQ ID NO: 46) and loop FG consists of amino acids 75-81 (KGGHRSN; SEQ ID NO: 47).

A Fibcon, FibconB or Tencon moiety, or target binding variants thereof, whether by themselves or linked to a heterologous moiety may be fused as described herein. Fn3 domains from other proteins, e.g., cell surface hormone and cytokine receptors, chaperonins, and carbohydrate-binding domains, may be conjugated as described herein.

FBS proteins or moieties are described, e.g., in WO 2010/093627, WO 2011/130324, WO 2009/083804, WO 2009/133208, WO 02/04523, WO 2012/016245, WO 2009/023184, WO 2010/051310, WO 2011/020033, WO 2011/051333, WO 2011/051466, WO 2011/092233, WO 2011/100700, WO 2011/130324, WO 2011/130328, WO 2011/137319, WO 2010/051274, WO 2009/086116, WO 09/058379, WO 2013/067029 WO 2012/016245, WO 2014/120891 and WO 2014/043344 (all of which are specifically incorporated by reference herein): any of the FBS proteins or moieties described in these publications may be used as described herein.

In certain embodiments, a protein comprises at least 2 FBS moieties, e.g., the protein comprises a multivalent FBS moiety. For example, a multivalent FBS may comprise 2, 3 or more FBS moieties, e.g., ¹⁰Fn3 domains, that are covalently associated. In exemplary embodiments, the FBS moiety is a bispecific or dimeric protein comprising two ¹⁰Fn3 domains.

The FBS moieties, e.g., ¹⁰Fn3 domains, in a multivalent protein may be connected by a polypeptide linker. Exemplary polypeptide linkers include polypeptides having from 1-20, 1-15, 1-10, 1-8, 1-5, 1-4, 1-3, or 1-2 amino acids. Suitable linkers for joining the ¹⁰Fn3 domains are those which allow the separate domains to fold independently of each other forming a three dimensional structure that permits high affinity binding to a target molecule. Specific examples of suitable linkers include glycine-serine based linkers, glycine-proline based linkers, proline-alanine based linkers as well as any other linkers described herein. In some embodiments, the linker is a glycine-proline based linker. These linkers comprise glycine and proline residues and may be between 3 and 30, 10 and 30, and 3 and 20 amino acids in length. Examples of such linkers include GPG, GPGPGPG (SEQ ID NO: 48) and GPGPGPGPGPG (SEQ ID NO: 49). In some embodiments, the linker is a proline-alanine based linker. These linkers comprise proline and alanine residues and may be between 3 and 30, 10 and 30, 3 and 20 and 6 and 18 amino acids in length. Examples of such linkers include PAPAPA (SEQ ID NO: 50), PAPAPAPAPAPA (SEQ ID NO: 51) and PAPAPAPAPAPAPAPAPA (SEQ ID NO: 52). In some embodiments, the linker is a glycine-serine based linker. These linkers comprise glycine and serine residues and may be between 8 and 50, 10 and 30, and 10 and 20 amino acids in length. Examples of such linkers include GSGSGSGSGS ((GS) 5; SEQ ID NO: 53), GSGSGSGSGSGS ((GS) 6; SEQ ID NO: 54), GSGSGSGSGSGSGSGSGSGS ((GS) 10; SEQ ID NO: 55), GGGGSGGGGSGGGGS ((G4S) 4; SEQ ID NO: 56), GGGGSGGGGSGGGGSGGGGSGGGGS ((G4S) 5; SEQ ID NO: 57), and GGGGSGGGGSGGGSG (SEQ ID NO: 58). In exemplary embodiments, the linker does not contain any Asp-Lys (DK) pairs.

### PmXn Moieties, e.g., Stabilizing Moieties

In certain embodiments, an FBS, e.g., a ¹⁰Fn3 moiety, is linked at its C-terminus to a moiety consisting of PmXn, wherein P is a proline, X is any amino acid, m is an integer that is at least 1 and n is 0 or an integer that is at least 1, and P is N-terminal to X. The PmXn moiety may be linked directly to the C-terminal amino acid of a ¹⁰Fn3 moiety, e.g., to its 94^{th} amino acid (based on amino acid numbering of SEQ ID NO: 1). The PmXn moiety may be linked via a peptide bond to the 94^{th} amino acid of a ¹⁰Fn3 moiety. A PmXn moiety may be linked to a ¹⁰Fn3 moiety having an amino acid sequence that is homologous to that of SEQ ID NO: 1 or comprises, consists essentially of or consists of an amino acid sequence shown in Table 1 or 2. A single proline residue at the end of SEQ ID NO: 1 is referred to as “95Pro” or “Pro95” or “P95” or “95P”.

Exemplary ¹⁰Fn3 moieties linked to a PmXn moiety include the following:

(SEQ ID NO: 59)
LEVVAATPTSLLISWDAPAVTVRYYRITYGETGGNSPVQEFTVPGSKSTA
TISGLKPGVDYTITVYAVTGRGDSPASSKPISINYRTPmXn
(SEQ ID NO: 60)
VSDVPRDLEVVAATPTSLLISWDAPAVTVRYYRITYGETGGNSPVQEFTV
PGSKSTATISGLKPGVDYTITVYAVTGRGDSPASSKPISINYRTPmXn

In PmXn, m may be 1, 2, 3 or more. For example, m may be 1-3 or m may be 1-2. “n” may be 0, 1, 2, 3 or more, e.g., n may be 1-3 or 1-2.

As further described herein, these ¹⁰Fn3 moieties may be modified to bind to a target (and form FBS moieties), by modifying the amino acid sequence of one or more loop and/or one or more β-strands. FBS moieties that are linked to PmXn are referred to herein as “modified FBS moieties”. Accordingly, provided herein are proteins comprising a FBS moiety comprising an amino acid sequence that is at least about 50%, 60%, 70%, 80%, 90%, or 95% identical to SEQ ID NO: 59 or 60, wherein the protein comprises PmXn, and wherein the FBS binds specifically to a target (other than through the RGD domain).

In PmXn, n may be 0, in which case, the C-terminal amino acid of the protein is Pm, e.g., P. In certain embodiments, n is not 0, and may be, e.g., 1, 2, 3, 4, 5, 6, 7, 8, 9, 10 or more. For example, n may be from 0-10, 0-5, 0-3, 1-10, 1-5, 1-3 or 1-2. However, more than 10 amino acids may be linked to the proline. For example, in a tandem FBS moiety or a FBS moiety fused to another polypeptide, the C-terminal amino acid of the FBS moiety may be linked to one or more prolines, and the last proline is linked to the second FBS moiety or to the heterologous moiety. Therefore, in certain embodiments, n may be an integer ranging from 0-100, 0-200, 0-300, 0-400, 0-500 or more.

In certain embodiments, PmXn comprises a cysteine. For example, the first amino acid after the proline may be a cysteine, and the cysteine may be the last amino acid in the molecule or the cysteine may be followed by one or more amino acids. The presence of a cysteine permits the conjugation of heterologous moieties to the FBS moiety, e.g., chemical moieties, e.g., PEG. Exemplary PmXn moieties comprising a cysteine include: PmCXn, wherein C is a cysteine. Another example is PmXn₁CXn₂, wherein n₁ and n₂ are independently 0 or an integer that is at least 1. For example, n₁ may be 1 and n₂ may be 1, 2, 3, 4 or 5.

Exemplary PmXn moieties include those listed in Table 3.

TABLE 3
Exemplary PmXn moieties

| Moieties with 1 proline | Moieties with 2 prolines |
|---|---|
| P | PP |
| PI | PPI |
| PC | PPC |
| PID | PPID (SEQ ID NO: 114) |
| PIE | PPIE (SEQ ID NO: 115) |
| PIDK (SEQ ID NO: 61) | PPIDK (SEQ ID NO: 62) |
| PIEK (SEQ ID NO: 63) | PPIEK (SEQ ID NO: 64) |
| PIDKP (SEQ ID NO: 65) | PPIDKP (SEQ ID NO: 66) |
| PIEKP (SEQ ID NO: 67) | PPIEKP (SEQ ID NO: 68) |
| PIDKPS (SEQ ID NO: 69) | PPIDKPS (SEQ ID NO: 70) |
| PIEKPS (SEQ ID NO: 71) | PPIEKPS (SEQ ID NO: 72) |
| PIDKPC (SEQ ID NO: 73) | PPIDKPC (SEQ ID NO: 74) |
| PIEKPC (SEQ ID NO: 75) | PPIEKPC (SEQ ID NO: 76) |
| PIDKPSQ (SEQ ID NO: 77) | PPIDKPSQ (SEQ ID NO: 78) |
| PIEKPSQ (SEQ ID NO: 79) | PPIEKPSQ (SEQ ID NO: 80) |
| PIDKPCQ (SEQ ID NO: 81) | PPIDKPCQ (SEQ ID NO: 82) |
| PIEKPCQ (SEQ ID NO: 83) | PPIEKPCQ (SEQ ID NO: 84) |
| PHHHHHH (SEQ ID NO: 85) | PPHHHHHH (SEQ ID NO: 86) |
| PCHHHHHH (SEQ ID NO: 87) | PPCHHHHHH (SEQ ID NO: 88) |

Any of the PmXn moieties, e.g., those shown in Table 3 may be followed by a histidine tail, e.g., 6xHis tag, or other tag. This does not exclude that a histidine tail may be included in PmXn.

The addition of a PmXn moiety to an FBS moiety enhances one or more characteristics of the FBS moiety. For example, as shown in the Examples, it enhances the thermostability of a ¹⁰Fn3 moiety relative to the moiety that is not linked to a PmXn moiety. The improvement of thermostability is expected to improve other desirable properties such as solubility, proper folding and expression level. For example, as shown in the Example, the presence of a PmXn moiety at the C-terminus of an FBS enhances the solubility of the FBS relative to the FBS that is not linked to a PmXn moiety.

Thus, in certain embodiments, the Tm of an FBS, e.g., a ¹⁰Fn3, moiety is enhanced by at least 1, 2, 3, 4, 5, 6, 7, 8, 9, 10, 11, 12, 13, 14, or 15° C. relative to the FBS moiety that is not linked to a PmXn moiety. For example, the Tm may be increased from 1-30, 1-25, 1-20, 1-15, 1-10, or 1-5° C. relative to the FBS moiety that is not linked to a PmXn moiety. Tm may be measured by Thermal Scanning Fluorescence (TSF), e.g., as follows. Protein samples, e.g., HTPP samples, are normalized to 0.2 mg/ml in PBS. 1 μl of SYPRO® orange dye diluted 1:40 with PBS is added to 25 μl of each sample and the plate is sealed with a clear 96-well microplate adhesive seal. Samples are scanned using a BioRad RT-PCR machine by ramping the temperature from 25° C.-95° C., at a rate of 2 degrees per minute. The data is analyzed using BioRad CFX manager 2.0 software. Tm may also be measured by Differential Scanning calorimetry (DSC) as follows. A 0.5 mg/ml solution is scanned in a VP-Capillary Differential Scanning calorimeter (GE Microcal) by ramping the temperature from 15° C. to 110° C. at a rate of 1 degree per minute under 70 p.s.i pressure. The data is analyzed versus a control run of the appropriate buffer using a best fit using Origin Software (OriginLab Corp).

In certain embodiments, the solubility of an FBS moiety is enhanced by linking it to a PmXn moiety. Such a molecule may exist at a concentration of at least 10 mg/ml, 20 mg/ml, 30 mg/ml, 40 mg/ml, 50 mg/ml, 60 mg/ml, 70 mg/ml, 80 mg/ml, 90 mg/ml or 100 mg/ml.

### PKE Moieties

FBS moieties may be linked to PKE moieties to extend the half-life of the FBS moieties. Exemplary PKE moieties include human serum albumin; proteins that bind to human serum albumin (e.g., an FBS binding to HSA or ABD); Fc; or any portion or variant thereof; and PEGs. These moieties may be linked N-terminal or C-terminal to the PmXn moiety and/or the FBS.

### Cysteine Conjugated Labels and Therapeutics

In certain embodiments, an FBS moiety linked to a PmXn moiety (and referred to a “modified FBS moiety”), wherein at least one or more the amino acids “X” is a cysteine, is linked through the one or more cysteines, to a heterologous moiety, such as a labeling moiety, a biologically active moiety (e.g., a therapeutic agent) or a binding moiety.

FBS moieties described herein may be conjugated through a C-terminal cysteine to a therapeutic agent to form an immunoconjugate such as an FBS-drug conjugate (FBS-DC; also “adnectin-drug conjugate”).

In an FBS-DC, the FBS is conjugated to a drug, with the FBS functioning as a targeting agent for directing the FBS-DC to a target cell expressing its antigen, such as a cancer cell. Preferably, the antigen is a tumor associated antigen, i.e., one that is uniquely expressed or over-expressed by the cancer cell. Once there, the drug is released, either inside the target cell or in its vicinity, to act as a therapeutic agent. For a review on the mechanism of action and use of drug conjugates as used with antibodies, e.g., in cancer therapy, see Schrama et al., Nature Rev. Drug Disc., 5:147 (2006).

Suitable therapeutic agents for use in drug conjugates include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In an FBS-DC, the FBS and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val (SEQ ID NO: 89), Ala-Asn-Val, Val-Leu-Lys, Ala-Ala-Asn, Cit-Cit, Val-Lys, Lys, Cit, Ser, or Glu. The FBS-DCs can be prepared according to methods similar to those described in U.S. Pat. Nos. 7,087,600; 6,989,452; and 7,129,261; PCT Publication Nos. WO 02/096910; WO 07/038658; WO 07/051081; WO 07/059404; WO 08/083312; and WO 08/103693; U.S. Patent Publication Nos. 2006/0024317; 2006/0004081; and 2006/0247295; the disclosures of which are incorporated herein by reference. A linker can itself be linked, e.g., covalently linked, e.g., using maleimide chemistry, to a cysteine of the PmXn moiety, wherein at least one X is a cysteine. For example, a linker can be covalently linked to an FBS-PmXn, wherein at least one X is a cysteine. For example, a linker can be linked to an FBS-PmCn, wherein P is a proline, C is a cysteine, and m and n are integers that are at least 1, e.g., 1-3.

Ligation to a cysteine can be performed as known in the art using maleimide chemistry (e.g., Imperiali, B. et al., Protein Engineering: Nucleic Acids and Molecular Biology, Vol. 22, pp. 65-96, Gross, H.J., ed. (2009)). For attaching a linker to a cysteine on an FBS, the linker may, e.g., comprise a maleinimido moiety, which moiety then reacts with the cysteine to form a covalent bond. In certain embodiments, the amino acids surrounding the cysteine are optimized to facilitate the chemical reaction. For example, a cysteine may be surrounded by negatively charged amino acid for a faster reaction relative to a cysteine that is surrounded by a stretch of positively charged amino acids (EP 1074563).

For cancer treatment, the drug preferably is a cytotoxic drug that causes death of the targeted cancer cell. Cytotoxic drugs that can be used in FBS-DCs include the following types of compounds and their analogs and derivatives:
(a) enediynes such as calicheamicin (see, e.g., Lee et al., J. Am. Chem. Soc., 109:3464, 3466 (1987)) and uncialamycin (see, e.g., Davies et al., WO 2007/038868 A2 (2007) and Chowdari et al., U.S. Pat. No. 8,709,431 B2 (2012));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Publication No. 2014/0227295 A1;
(c)CC-1065 and duocarmycin (see, e.g., Boger, U.S. Pat. No. 6,5458,530 B1 (2003); Sufi et al., U.S. Pat. No. 8,461,117 B2 (2013); and Zhang et al., U.S. Publication No. 2012/0301490 A1 (2012));
(d) epothilones (see, e.g., Vite et al., U.S. Publication No. 2007/0275904 A1 (2007) and U.S. Pat. No. RE42,930 E (2011));
(e) auristatins (see, e.g., Senter et al., U.S. Pat. No. 6,844,869 B2 (2005) and Doronina et al., U.S. Pat. No. 7,498,298 B2 (2009));
(f) pyrrolobenzodiazepine (PBD) dimers (see, e.g., Howard et al., U.S. Publication Nos. 2013/0059800 A1 (2013) and 2013/0028919 A1 (2013); and WO 2013/041606 A1 (2013)); and
(g) maytansinoids such as DM1 and DM4 (see, e.g., Chari et al., U.S. Pat. No. 5,208,020 (1993) and Amphlett et al., U.S. Pat. No. 7,374,762 B2 (2008)).

In certain embodiments, an FBS-PmXn, wherein at least one X is a cysteine, is linked to a labeling or detectable moiety for use, e.g., in vitro or in vivo detection or imaging.

Detectable labels can be any of the various types used currently in the field of in vitro diagnostics, including particulate labels including metal sols such as colloidal gold, isotopes such as [I²⁵ or Tc⁹⁹ presented for instance with a peptidic chelating agent of the N₂S₂, N₃S or N₄ type, chromophores including fluorescent markers, biotin, luminescent markers, phosphorescent markers and the like, as well as enzyme labels that convert a given substrate to a detectable marker, and polynucleotide tags that are revealed following amplification such as by polymerase chain reaction. A biotinylated antibody would then be detectable by avidin or streptavidin binding. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro {1,2-dioxetane-3,2′-(5′-chloro) tricyclo {3.3.1.1 3,7}decan}-4-yl) phenyl phosphate (CSPD), as well as CDP and CDP-STAR® or other luminescent substrates well-known to those in the art, for example the chelates of suitable lanthanides such as Terbium (III) and Europium (III).

Detectable moieties that may be used include radioactive agents, such as: radioactive heavy metals such as iron chelates, radioactive chelates of gadolinium or manganese, positron emitters of oxygen, nitrogen, iron, carbon, or gallium, ¹⁸F, ⁶⁰Cu, ⁶¹Cu, ⁶²Cu, ⁶⁴Cu, ¹²⁴I, ⁸⁶Y, ⁸⁹Zr, ⁶⁶Ga, ⁶⁷Ga, ⁶⁸Ga, ⁴⁴Sc, ⁴⁷Sc, ¹¹C, ¹¹¹In, ^{114m}In, ¹¹⁴In, ¹²⁵I, ¹²⁴I, ¹³¹I, ¹²³I, ¹³¹I, ¹²³I, ³²Cl, ³³Cl, ³⁴Cl, ⁷⁴Br, ⁷⁵Br, ⁷⁶Br, ⁷⁷Br, ⁷⁸Br, ⁸⁹Zr, ¹⁸⁶Re, ¹⁸⁸Re, ⁸⁶Y, ⁹⁰Y, ¹⁷⁷Lu, ⁹⁹Tc, ²¹²Bi, ²¹³Bi, ²¹²Pb, ²²⁵Ac, or ¹⁵³Sm.

The detection means is determined by the chosen label. Appearance of the label or its reaction products can be achieved using the naked eye, in the case where the label is particulate and accumulates at appropriate levels, or using instruments such as a spectrophotometer, a luminometer, a fluorometer, and the like, all in accordance with standard practice.

A detectable moiety may be linked to a cysteine according to methods known in the art. When the detectable moiety is a radioactive agent, e.g., those described further herein, the detectable moiety is linked to an FBS through a chelating agent that is reactive with cysteines, such as a maleimide containing chelating agent, such as maleimide-NODAGA or maleimide-DBCO. Maleimide-NODAGA or maleimide-DBCO can be reacted with a cysteine on the C-terminus of an FBS (e.g., through the PmXn moiety, wherein at least one X is a cysteine), to yield FBS-NODAGA or FBS-DBCO, respectively. Any one of the following chelating agents may be used provided that it comprises, or can be modified to comprise, a reactive moiety that reacts with cysteines: DFO, DOTA and its derivatives (CB-DO2A, 3p-C-DEPA, TCMC, Oxo-DO3A), TE2A, CB-TE2A, CB-TE1A1P, CB-TE2P, MM-TE2A, DM-TE2A, diamsar and derivatives, NODASA, NODAGA, NOTA, NETA, TACN-TM, DTPA, 1B4M-DTPA, CHX-A″-DTPA, TRAP (PRP9), NOPO, AAZTA and derivatives (DATA), H₂dedpa, H₄octapa, H₂azapa, H₅decapa, H₆phospa, HBED, SHBED, BPCA, CP256, PCTA, HEHA, PEPA, EDTA, TETA, and TRITA based chelating agents, and close analogs and derivatives thereof.

In certain embodiments, an FBS is labeled with a PET tracer and used as an in vivo imaging agent. For example, an FBS may be labeled with the PET tracer 64Cu. 64Cu may be linked to an FBS with a C-terminal cysteine with a chelating agent, such as maleimide-NODAGA.

### Exemplary Molecules

In certain embodiments, a protein comprises (i) an FBS moiety comprising an amino acid sequence that is at least about 50%, 60%, 70%, 75%, 80%, 85%, 90%, 95%, 97%, 98%, or 99% identical to any one of SEQ ID NOs: 1-16; and (ii) PmXn, wherein P is a proline, X is any amino acid, m is an integer of at least 1 and n is 0 or an integer of at least 1, wherein the protein binds specifically to a target (e.g., with a K_{d} of less than 500 nM, 100 nM, 50 nM, 10 nM, 5 nM, 1 nM, 500 μM, 100 μM or less, as determined, e.g., by Surface Plasmon Resonance (SPR), such as Biacore) and wherein the PmXn moiety improves at least one property of the FBS moiety relative to a protein consisting of the unmodified FBS moiety.

In certain embodiments, an enhanced property conferred by a PmXn moiety is enhanced protein stability, e.g., an increase in melting temperature (Tm) of at least 1° C., 2° C., 3° C., 4° C., 5° C., 6° C., 7° C., 8° C., 9° C., 10° C., 11° C., 12° C., 13° C., 14° C., 15° C., 1-15° C., 2-15° C., 3-15° C., 5-15° C., 5-15° C., 1-10° C., 2-10° C., 3-10° C., 4-10° C. or 5-10° C. Tm may be determined, e.g., by Thermal Scanning Fluorescence (TSF) or Differential scanning calorimetry (DSC). “Enhanced Tm” refers to a statistically significant enhancement of the Tm.

In certain embodiments, a protein comprising an FBS moiety and a PmXn moiety is present in a composition, e.g., a pharmaceutical composition, at a concentration of at least 10 mg/ml, 20 mg/ml, 30 mg/ml, 40 mg/ml, 50 mg/ml, 60 mg/ml, 70 mg/ml, 80 mg/ml, 90 mg/ml or 100 mg/ml.

In certain embodiments, a protein comprising an FBS moiety and a PmXn moiety is present in a composition, e.g., a pharmaceutical composition, mostly as a monomer, e.g., at least 80%, 85%, 90%, 95%, 98%, or 99% of the protein in the composition is in monomeric form. Degree of monomericity of a protein solution may be determined by Size Exclusion Chromatography (SEC), e.g., by using a Superdex column (GE Healthcare) on an Agilent 1100 or 1200 HPLC system with UV detection at A214 nm and A₂₈₀ nm and with fluorescence detection (excitation=280 nm, emission=350 nm). A buffer of 100 mM sodium sulfate, 100 mM sodium phosphate, 150 mM sodium chloride, (e.g., pH 6.8) at an appropriate flow rate of the SEC column may be employed. Gel filtration standards (Bio-Rad Laboratories, Hercules, CA) are used for molecular weight calibration.

In certain embodiments, a protein comprising an FBS moiety and a PmXn moiety has a biological activity that is as least as strong as that of the unmodified FBS moiety. Biological activity can be binding affinity to a target or a biological activity in an assay, e.g., the ability to destroy tumor cells. In certain embodiments, the biological activity of the protein is within 5%, 10%, 25%, 50%, 100%, 2 fold or more of the activity of the unmodified FBS moiety.

A protein comprising an FBS and a PmXn moiety may also comprise a combination of the above characteristics. For example, a protein may be soluble at concentrations of up to 50 mg/ml, be present at least 90% in monomeric form, and/or have a biological activity that is at least as potent as that of the unmodified FBS.

When referring to an enhanced property, the enhancement is a statistically significant enhancement.

### Nucleic Acid-Protein Technology

One way to rapidly make and test FBS domains with specific binding properties is the nucleic acid-protein technology of Adnexus, a Bristol-Myers Squibb Company. Such in vitro expression and tagging technology, termed Profusion, that exploits nucleic acid-protein fusions (RNA- and DNA-protein fusions) may be used to identify novel polypeptides and amino acid motifs that are important for binding to proteins. Nucleic acid-protein technology is a technology that covalently couples a protein to its encoding genetic information. For a detailed description of the RNA-protein technology and fibronectin-based scaffold protein library screening methods see Szostak et al., U.S. Pat. Nos. 6,258,558; 6,261,804; 6,214,553; 6,281,344; 6,207,446; 6,518,018; PCT Publication Nos. WO 00/34784; WO 01/64942; WO 02/032925; and Roberts et al., Proc Natl. Acad. Sci., 94:12297-12302 (1997), herein incorporated by reference.

### Vectors and Polynucleotides Embodiments

Nucleic acids encoding any of the various proteins comprising an FBS moiety a PmXn moiety disclosed herein may be synthesized chemically, enzymatically or recombinantly. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA, 100 (2): 438-442 (Jan. 21, 2003); Sinclair et al., Protein Expr. Purif., 26 (1): 96-105 (October 2002); Connell, N.D., Curr. Opin. Biotechnol., 12 (5): 446-449 (October 2001); Makrides et al., Microbiol. Rev., 60 (3): 512-538 (September 1996); and Sharp et al., Yeast, 7 (7): 657-678 (October 1991).

General techniques for nucleic acid manipulation are described for example in Sambrook et al., Molecular Cloning: A Laboratory Manual, Second Edition, Vols. 1-3, Cold Spring Harbor Laboratory Press (1989), or Ausubel, F. et al., Current Protocols in Molecular Biology, Green Publishing and Wiley-Interscience, New York (1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants are additionally incorporated.

The proteins described herein may be produced recombinantly not only directly, but also as a polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process a native signal sequence, the signal sequence is substituted by a prokaryotic signal sequence selected, for example, from the group of the alkaline phosphatase, penicillinase, lpp, or heat-stable enterotoxin II leaders. For yeast secretion the native signal sequence may be substituted by, e.g., the yeast invertase leader, a factor leader (including Saccharomyces and Kluyveromyces alpha-factor leaders), or acid phosphatase leader, the C. albicans glucoamylase leader, or the signal described in PCT Publication No. WO 90/13646. In mammalian cell expression, mammalian signal sequences as well as viral secretory leaders, for example, the herpes simplex gD signal, are available. The DNA for such precursor regions may be ligated in reading frame to DNA encoding the protein.

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2u plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC® No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC® 20,622 or 38,626) are complemented by known plasmids bearing the Leu2 gene.

Expression and cloning vectors usually contain a promoter that is recognized by the host organism and is operably linked to the nucleic acid encoding the protein. Promoters suitable for use with prokaryotic hosts include the phoA promoter, beta-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the protein. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT (SEQ ID NO: 109) region where N may be any nucleotide. At the 3′ end of most eukaryotic genes is an AATAAA (SEQ ID NO: 110) sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP Patent Publication No. 73,657 and PCT Publication Nos. WO 2011/124718 and WO 2012/059486. Yeast enhancers also are advantageously used with yeast promoters.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the ACTIN® promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature, 297:598-601 (1982) on expression of human β-interferon cDNA in mouse cells under the control of a thymidine kinase promoter from herpes simplex virus. Alternatively, the rous sarcoma virus long terminal repeat can be used as the promoter.

Transcription of a DNA encoding a protein by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature, 297:17-18 (1982) on enhancing elements for activation of eukaryotic promoters. The enhancer may be spliced into the vector at a position 5′ or 3′ to the polypeptide-encoding sequence, but is preferably located at a site 5′ from the promoter.

Expression vectors used in eukaryotic host cells (e.g., yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide. One useful transcription termination component is the bovine growth hormone polyadenylation region. See WO 94/11026 and the expression vector disclosed therein.

The recombinant DNA can also include any type of protein tag sequence that may be useful for purifying the proteins. Examples of protein tags include but are not limited to a histidine tag, a FLAG® tag, a myc tag, an HA tag, or a GST tag. Appropriate cloning and expression vectors for use with bacterial, fungal, yeast, and mammalian cellular hosts can be found in Cloning Vectors: A Laboratory Manual, Elsevier, New York (1985), the relevant disclosure of which is hereby incorporated by reference.

The expression construct may be introduced into the host cell using a method appropriate to the host cell, as will be apparent to one of skill in the art. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent).

Suitable host cells include prokaryotes, yeast, mammalian cells, or bacterial cells. Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow et al. (Bio/Technology, 6:47 (1988)). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified proteins are prepared by culturing suitable host/vector systems to express the recombinant proteins. The FBS protein is then purified from culture media or cell extracts.

### Protein Production

Host cells are transformed with the herein-described expression or cloning vectors for protein production and cultured in conventional nutrient media modified as appropriate for inducing promoters, selecting transformants, or amplifying the genes encoding the desired sequences.

The host cells used to produce the proteins may be cultured in a variety of media. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enzymol., 58:44 (1979), Barnes et al., Anal. Biochem., 102:255 (1980), U.S. Pat. Nos. 4,767,704; 4,657,866; 4,927,762; 4,560,655; or 5,122,469; PCT Publication Nos. WO 90/03430; WO 87/00195; or U.S. Pat. No. RE30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Proteins disclosed herein can also be produced using cell-free translation systems. For such purposes the nucleic acids encoding the protein must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Proteins can also be produced by chemical synthesis (e.g., by the methods described in Solid Phase Peptide Synthesis, Second Edition, The Pierce Chemical Co., Rockford, IL (1984)). Modifications to the protein can also be produced by chemical synthesis.

The proteins disclosed herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, proteins may be exchanged into different buffers and/or concentrated by any of a variety of methods known to the art, including, but not limited to, filtration and dialysis.

The purified protein is preferably at least 85% pure, more preferably at least 95% pure, and most preferably at least 98% or 99% pure. Regardless of the exact numerical value of the purity, the protein is sufficiently pure for use as a pharmaceutical product.

### Exemplary Uses

Modified FBS proteins may be used for any purpose for which FBS proteins that are not modified by the addition of a PmXn moiety can be used.

In one aspect, the application provides proteins comprising a modified FBS moiety that is useful in the treatment of disorders. The diseases or disorders that may be treated will be dictated by the binding specificity of the FBS moiety. As described herein, modified FBS moieties may be designed to bind to any target of interest. Exemplary targets include, for example, TNF-alpha, VEGFR2, PCSK9, IL-23, EGFR and IGF1R. Merely as an example, modified FBS moieties that bind to TNF-alpha may be used to treat autoimmune disorders such as rheumatoid arthritis, inflammatory bowel disease, psoriasis, and asthma. Modified FBS proteins described herein may also be used for treating cancer.

In certain embodiments, a method for treating a subject having a disease, e.g., cancer, comprises administering to the subject a modified FBS-drug conjugate.

Provided herein are methods for administering proteins to a subject. In some embodiments, the subject is a human. In some embodiments, the proteins are pharmaceutically acceptable to a mammal, in particular a human. A “pharmaceutically acceptable” composition refers to a composition that is administered to an animal without significant adverse medical consequences. Examples of pharmaceutically acceptable compositions include compositions comprising FBS moieties that lack the integrin-binding domain (RGD) and compositions that are essentially endotoxin or pyrogen free or have very low endotoxin or pyrogen levels.

Other uses of the modified FBS proteins described herein include their use in in vitro or in vivo detection assays. For example, they may be used for detecting a target molecule in a sample. A method may comprise contacting the sample with a modified FBS described herein, wherein said contacting is carried out under conditions that allow FBS-target complex formation; and detecting said complex, thereby detecting said target in said sample. Detection may be carried out using any art-recognized technique, such as, e.g., radiography, immunological assay, fluorescence detection, mass spectroscopy, or surface plasmon resonance. The sample may be from a human or other mammal. For diagnostic purposes, appropriate agents are detectable labels that include radioisotopes, for whole body imaging, and radioisotopes, enzymes, fluorescent labels and other suitable antibody tags for sample testing.

In certain embodiments, the modified FBS described herein are useful in a variety of diagnostic and imaging applications. In certain embodiments, a modified FBS is labeled with a moiety that is detectable in vivo and such labeled FBS may be used as in vivo imaging agents, e.g., for whole body imaging. For example, in one embodiment, a method for detecting a tumor comprising a given antigen in a subject comprises administering to the subject a modified FBS linked to a detectable label, and following an appropriate time, detecting the label in the subject.

An FBS imaging agent may be used to diagnose a disorder or disease associated with increased levels of a given antigen, for example, a cancer in which a tumor selectively overexpresses the antigen. In a similar manner, a modified FBS that binds specifically to a given antigen can be used to monitor antigen levels in a subject being treated for a condition associated with the antigen. The modified FBS may be used with or without modification, and may be labeled by covalent or non-covalent attachment of a detectable moiety.

### Formulation and Administration

The application further provides pharmaceutically acceptable compositions comprising the proteins described herein, wherein the composition is essentially endotoxin and/or pyrogen free.

Therapeutic formulations comprising proteins are prepared for storage by mixing the described proteins having the desired degree of purity with optional physiologically acceptable carriers, excipients or stabilizers (Osol, A., ed., Remington's Pharmaceutical Sciences, 16^{th} Edition (1980)), in the form of aqueous solutions, lyophilized or other dried formulations. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween, PLURONIC® or polyethylene glycol (PEG).

The formulations herein may also contain more than one active compounds as necessary for the particular indication being treated, preferably those with complementary activities that do not adversely affect each other. Such molecules are suitably present in combination in amounts that are effective for the purpose intended.

The proteins may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Osol, A., ed., Remington's Pharmaceutical Sciences, 16^{th} Edition (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the proteins described herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

While the skilled artisan will understand that the dosage of each protein will be dependent on the identity of the protein, the preferred dosages can range from about 10 mg/square meter to about 2000 mg/square meter, more preferably from about 50 mg/square meter to about 1000 mg/square meter.

For therapeutic applications, the proteins are administered to a subject, in a pharmaceutically acceptable dosage form. They can be administered intravenously as a bolus or by continuous in over a period of time, by intramuscular, subcutaneous, intra-articular, intrasynovial, intrathecal, oral, topical, or inhalation routes. The protein may also be administered by intratumoral, peritumoral, intralesional, or perilesional routes, to exert local as well as systemic therapeutic effects. Suitable pharmaceutically acceptable carriers, diluents, and excipients are well known and can be determined by those of skill in the art as the clinical situation warrants. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose. The methods of the present invention can be practiced in vitro, in vivo, or ex vivo.

Administration of proteins, and one or more additional therapeutic agents, whether co-administered or administered sequentially, may occur as described above for therapeutic applications. Suitable pharmaceutically acceptable carriers, diluents, and excipients for co-administration will be understood by the skilled artisan to depend on the identity of the particular therapeutic agent being co-administered.

When present in an aqueous dosage form, rather than being lyophilized, the protein typically will be formulated at a concentration of about 0.1 mg/ml to 100 mg/ml, although wide variation outside of these ranges is permitted. For the treatment of disease, the appropriate dosage of proteins will depend on the type of disease to be treated, the severity and course of the disease, whether the proteins are administered for preventive or therapeutic purposes, the course of previous therapy, the patient's clinical history and response to the fusion, and the discretion of the attending physician. The protein is suitably administered to the patient at one time or over a series of treatments.

### Exemplary Embodiments

1. An isolated fibronectin based scaffold (FBS) protein that specifically binds to a target, wherein the FBS is a non-naturally occurring FBS, and wherein the FBS is linked at its C-terminus to a moiety consisting of the amino acid sequence PmXn, wherein P is proline, X is any amino acid, m is an integer that is at least 1 and n is 0 or an integer that is at least 1, and wherein the PmXn moiety provides an enhanced property to the FBS protein relative to the FBS protein that is not linked to the PmXn moiety.
2. The isolated FBS protein of embodiment 1, wherein the moiety consists of P (m is 1 and n is 0).
3. The isolated FBS protein of embodiment 1, wherein the moiety consists of PP (m is 2 and n is 0).
4. The isolated FBS protein of embodiment 1, wherein the moiety consists of PmXn, wherein n is 1-150.
5. The isolated FBS protein of embodiment 1, wherein the moiety consists of PmXn, wherein n is 1-10.
6. The isolated FBS protein of embodiment 1, wherein the moiety consists of PmXn, wherein n is 1-5.
7. The isolated FBS protein of embodiment 1, wherein the moiety consists of PI, PC, PID, PIE, PIDK (SEQ ID NO: 61), PIEK (SEQ ID NO: 63), PIDKP (SEQ ID NO: 65), PIEKP (SEQ ID NO: 67), PIDKPS (SEQ ID NO: 69), PIEKPS (SEQ ID NO: 71), PIDKPC (SEQ ID NO: 73), PIEKPC (SEQ ID NO: 75), PIDKPSQ (SEQ ID NO: 77), PIEKPSQ (SEQ ID NO: 79), PIDKPCQ (SEQ ID NO: 81), PIEKPCQ (SEQ ID NO: 83), PHHHHHH (SEQ ID NO: 87) or PCHHHHHH (SEQ ID NO: 86).
8. The isolated FBS protein of any one of embodiments 1-7, wherein the FBS protein is an Fn3 protein.
9. The isolated FBS protein of embodiment 8, wherein the Fn3 protein is a ¹⁰Fn3 protein.
10. The isolated FBS protein of embodiment 9, wherein the ¹⁰Fn3 protein is a human ¹⁰Fn3 protein.
11. The isolated FBS protein of any one of embodiments 1-10, comprising an amino acid sequence that is at least 50% identical to SEQ ID NO: 1.
12. The isolated FBS protein of any one of embodiments 1-11, comprising at least one amino acid mutation in at least one loop or one scaffold region.
13. The isolated FBS protein of any one of embodiments 1-12, comprising the amino acid sequence VSDVPRDLEVVAA (X)ₙLLISW (X)ᵥYRITY(X)_{w}FTV(X)ₓATISGL(X)_{y}YTITVY A(X)_{z}ISINYRT (SEQ ID NO: 9), wherein (X)ᵤ, (X)ᵥ, (X)_{w}, (X)ₓ, (X)_{y} and (X)_{z} consist of the wild-type amino acid sequence (SEQ ID NO: 1) or comprise at least one amino acid difference with the corresponding wild-type sequence, and the sequence optionally comprises from 1-10 scaffold, N-terminal and/or C-terminal mutations.
14. The isolated FBS protein of any one of embodiments 1-13, comprising an amino acid sequence that is at least 50% identical to SEQ ID NO: 1, and wherein the C-terminal amino acid residue of the FBS protein is fused to a moiety consisting of PmXn, wherein the FBS protein binds specifically to a target with a Kd of less than 500 nM, which target is not bound by a wild-type ¹⁰Fn3 molecule comprising SEQ ID NO: 1.
15. The isolated FBS protein of embodiment 14, comprising an amino acid sequence that is at least 70% identical to SEQ ID NO: 1.
16. The isolated FBS protein of any one of embodiments 1-15, wherein the moiety consisting of PmXn is linked to the C-terminal amino acid residue of the FBS protein through a peptide bond.
17. The isolated FBS protein of any one of embodiments 1-16, wherein PmXn is P or PC.
18. The isolated FBS protein of any one of embodiments 1-17, comprising an amino acid sequence that is at least 60% identical to SEQ ID NO: 1, and wherein the C-terminal amino acid residue of the FBS protein is linked to a proline through a peptide bond, and wherein the FBS protein binds specifically to a target with a Kd of less than 500 nM, which target is not bound by a wild-type ¹⁰Fn3 molecule comprising SEQ ID NO: 1.
19. The isolated FBS protein of any of embodiments 1-18, wherein at least one X of PmXn is a cysteine and wherein the cysteine
20. The isolated FBS protein of embodiment 19, wherein the cysteine is conjugated to a heterologous moiety.
21. The isolated FBS protein of embodiment 20, wherein the heterologous molecule is a detectable moiety.
22. The isolated FBS protein of embodiment 20 or 21, wherein the heterologous molecule is a drug moiety and the drug moiety and the FBS form an FBS-drug conjugate.
23. The isolated FBS protein of any one of embodiments 1-22, wherein the enhanced property conferred by the PmXn moiety is enhanced stability.
24. The isolated FBS protein of embodiment 23, wherein enhanced stability is an increase in Tm of at least 1° C., 2° C., 3° C., 4° C., 5° C. or more.

The following representative Examples contain important additional information, exemplification and guidance which can be adapted to the practice of this invention in its various embodiments and the equivalents thereof. These examples are intended to help illustrate the invention, and are not intended to, nor should they be construed to, limit its scope.

### EXAMPLES

### Example 1: Thermostability Enhancement by Adding a Proline at the C-Terminus of ¹⁰Fn3 Molecules

In nature, ¹⁰Fn3 is a part of a long string of fibronectin type III domains; the domain immediately downstream of ¹⁰Fn3 is named 11Fn3. The sequence below shows the wild-type sequences of the ¹⁰Fn3 (in italics) and 11Fn3 (in bold) domains and the junction between them (underlined).

(SEQ ID NO: 90)
VSDVPRDLEVVAATPTSLLISWDAPAVTVRYYRITYGETGGNSPVQEFTV
PGSKSTATISGLKPGVDYTITVYAVTGRGDSPASSKPISINYRTEIDKPS
QMQVTDVQDNSISVKWLPSSSPVTGYRVTTTPKNGPGPTKTKTAGPDQTE
MTIEGLQPTVEYVVSVYAQNPSGESQPLVQTAVT

Based on the crystal and NMR structures of ¹⁰Fn3 and of the sequence alignment between ¹⁰Fn3 and 11Fn3 (Dickinson et al., “Crystal structure of the tenth type III cell adhesion module of human fibronectin”, J. Mol. Biol., 36:1079-1092 (1994)), the first two residues of this sequence, RT, are part of the final beta strand of ¹⁰Fn3 (“strand G”), and the remainder of the sequence, EIDKPSQ (SEQ ID NO: 113), is a flexible linker between structured tenth and eleventh fibronectin type III domains.

The C-terminus of engineered protein domains based on ¹⁰Fn3 is often modified from the wild-type sequence to facilitate its cloning, expression, and purification. During a survey of different engineered C-termini of Adnectins it was found that a mutation from RTE to RTP has led to increased thermostability of several different Adnectins. Table 4 lists different C-termini used in this study.

TABLE 4
Wild-type linker between ¹⁰Fn3 and ¹¹Fn3 and the engineered Adnectin C-
terminal sequences that were compared in this study. The “short engineered C-terminus
with P and His-tag” and the “short engineered C-terminus with PC” contain the RTE to
RTP mutation.

| Descriptor | Sequence | Purpose | Comments |
|---|---|---|---|
| Wild-type junction | RTEIDKPSQ | natural human | contains RTE |
| between ¹⁰Fn3 and | (SEQ ID NO: | sequence | |
| ¹¹En3 | 91) | | |
| Long engineered | RTEIEKPSQH₆ | H₆ allows purification | contains RTE; DK->EK |
| C-terminus with | (SEQ ID NO: | by metal-chelate | mutation reduced |
| His-tag | 92) | chromatography | sensitivity to proteases |
| Short engineered | RTPH₆ | H₆ allows purification | contains RTP |
| C-terminus with P | (SEQ ID NO: | by metal-chelate | |
| and His-tag | 93) | chromatography | |
| Short engineered | RTEC | C allows site-specific | contains RTE; no |
| C-terminus with | (SEQ ID NO: | modification using | purification tag |
| EC | 94) | maleimide chemistry | |
| Short engineered | RTPC | C allows site-specific | contains RTP; no |
| C-terminus with | (SEQ ID NO: | modification using | purification tag |
| PC | 95) | maleimide chemistry | |

The effect of the mutation from RTE to RTP on thermostability of Adnectins was tested by comparing several pairs of Adnectins that differed in their C-termini. Table 5 describes the pairs of Adnectins used in this study, including their names, targets, and thermodynamic properties. Adnectin 1 is an Adnectin that binds to target X. All the Adnectins listed were selected from high-complexity libraries using PROfusion (mRNA-display) technology, and had their C-termini re-engineered by site-directed mutagenesis. Full protein sequences are set forth below.

TABLE 5
Clone name, C-terminus, target, and melting temperature (as determined by
differential scanning calorimetry at 0.5 mg/mL, in PBS, pH 7.4). Adnectins PRD-1414
and PRD-1417 were conjugated to 40 kDa, 2-branched PEG (NOF, Cat. #GL2-
400MA01)

| | | | | | Increase in |
|---|---|---|---|---|---|
| | | | | | Tₘ of RTP-over |
| Protein ID (RTE) | Protein ID (RTP) | | Tₘ | Tₘ | RTE-containing |
| C-terminus | C-terminus | Target | (RTE) | (RTP) | protein |
| ADX_2382_D09 | ADX_5484_A03 | PCSK9 | 84° C. | 87° C. | +3° C. |
| RTEIEKPSQH₆ | RTPH₆ | | | | |
| (SEQ ID NO: 92) | (SEQ ID NO: 93) | | | | |
| ADX_2987_H07 | ADX_5484_A04 | Myostatin | 56° C. | 57° C. | +1° C. |
| RTEIEKPSQH₆ | RTPH₆ | | | | |
| (SEQ ID NO: 92) | (SEQ ID NO: 93) | | | | |
| PRD-1414 | PRD-1417 | PCSK9 | 64° C. | 76° C. | +12° C. |
| RTEC-PEG | RTPC-PEG | | | | |
| (SEQ ID NO: 94) | (SEQ ID NO: 95) | | | | |
| Adnectin 1 | Adnectin 1 | X | 61° C. | 64° C., | +3° C., +11° C. |
| RTEIEKPSQH₆ | RTPH₆ | | | 72° C. | |
| (SEQ ID NO: 94) | (SEQ ID NO: 95) | | | | |

Amino acid sequences of proteins used in this Example:

ADX_2382_D09:
(SEQ ID NO: 96)
MGVSDVPRDLEVVAATPTSLLISWDAPAEGYGYYRITYGETGGNSPVQE
FTVPVSKGTATISGLKPGVDYTITVYAVEFDFPGAGYYHRPISINYRTE
IDKPSQHHHHHH*
ADX_5484_A03:
(SEQ ID NO: 97)
MGVSDVPRDLEVVAATPTSLLISWDAPAEGYGYYRITYGETGGNSPVQE
FTVPVSKGTATISGLKPGVDYTITVYAVEFDFPGAGYYHRPISINYRTP
HHHHHH*
ADX_2987_H07:
(SEQ ID NO: 97)
MGVSDVPRDLEVVAATPTSLLISWTLPHAGRAHYYRITYGETGGNSPVQ
EFTVPGRGVTATISGLKPGVDYTITVYAVTVTTTKVIHYKPISINYRTE
IDKPSQHHHHHH*
ADX_5484_A04:
(SEQ ID NO: 98)
MGVSDVPRDLEVVAATPTSLLISWTLPHAGRAHYYRITYGETGGNSPVQ
EFTVPGRGVTATISGLKPGVDYTITVYAVTVTTTKVIHYKPISINYRTP
HHHHHH*
PRD-1414:
(SEQ ID NO: 99)
MGVSDVPRDLEVVAATPTSLLISWDAPAEGYGYYRITYGETGGNSPVQE
FTVPVSKGTATISGLKPGVDYTITVYAVEFDFPGAGYYHRPISINYRTE
C*
PRD-1417:
(SEQ ID NO: 100)
MGVSDVPRDLEVVAATPTSLLISWDAPAEGYGYYRITYGETGGNSPVQE
FTVPVSKGTATISGLKPGVDYTITVYAVEFDFPGAGYYHRPISINYRTP
C*
The ″*″ in each sequence above denotes the stop codon, and the C-terminus of each Adnectin.

### Adnectin

The N-terminal M was removed during bacterial expression of each protein. The C residues in purified PRD-1414 and PRD-1417 protein were conjugated to PEG before characterization of each protein.

As shown in Table 5, Adnectins with C-termini that contain a proline in place of the wild-type glutamic acid at the C-terminus (RTE to RTP mutation) show a higher thermostability. This increase in stability was observed for several examples when a longer C-terminus modeled on the natural linker between human ¹⁰Fn3 and 11Fn3 was replaced with a shorter engineered C-terminus containing only the proline and a hexahistidine purification tag. Enhanced thermostability was also observed in a ¹⁰Fn3 protein having a short engineered C-terminus and a polyethylene glycol (PEG) upon the addition of a C-terminal proline. The increase in stability may be attributed to the presence of a proline in this specific position.

### Example 2: Stabilization of a Second PCSK9 Adnectin with a C-terminal Proline

This Example shows that the thermostability of another PCSK9 adnectin molecule is also enhanced by the addition of a C-terminal proline.

In this Example, the C-terminus of pegylated (40 kDa branched PEG) PCSK9 adnectin ADX_2013_E01 was modified from NYRTEIEKPCQ (SEQ ID NO: 101) to NYRTPC (SEQ ID NO: 102) and thermostability measured by TSF. The amino acid sequence of this adnectin is provided in WO 2011/130354. The results indicate that the pegylated adnectin with the NYRTEIEKPCQ (SEQ ID NO: 101)C-terminus has a Tm of 70° C., whereas the pegylated adnectin with the NYRTPC (SEQ ID NO: 102)C-terminus has a Tm of 76° C.

Thus, the thermostability of this PCSK9 adnectin is enhanced by 6° C. by the presence of a C-terminal proline.

### Example 3: Comparison of Various C-termini on Thermostability

This Example shows a comparison of the thermostability of adnectins with or without a proline at their C-terminus as well as adnectins having 2 prolines at their C-terminus.

Two of the adnectins described in Example 1 (ADX_2392_D09 binding to PCSK9 and ADX_2987_H07 binding to myostatin) and one additional Adnectin binding to a different target (Adnectin 1, binding to target X) were modified at their C-terminus, as indicated in Table 6, and their thermostability and % monomer were determined. An Adnectin (“Adnectin 1”) to a different target (X) was also modified with the same C-terminal sequences. Thermostability was determined by TSF and % Monomer was determined by SEC.

TABLE 6
Percent monomer and thermostability of adnectins with various C-termini

| | | NYRTH₆ | NYRTPH₆ | NYRTPPH₆ |
|---|---|---|---|---|
| | | (SEQ ID | (SEQ ID | (SEQ ID |
| | NYRTEIEKPSQH₆ | NO: | NO: | NO: |
| Parent\C-term. | (SEQ ID NO: 103) | 104) | 105) | 106) |
| ADX_2392_D09 | >99% | >99% | >99% | 99% |
| PCSK9 | 84° C. | 83° C. | 87° C. | 87° C. |
| ADX_2987_H07 | 99% | >99% | >99% | >99% |
| Myostatin | 56° C. | 51° C. | 57° C. | 57° C. |
| Adnectin 1 | ~8% | >99% | >98% | >98% |
| Target X | 61° C. | 58° C. | 64° C., 72° C. | 64° C., 72° C. |

The results, which are set forth in Table 6, show that the identity of the C-terminus has no significant effect on the % monomer, however, it has an effect on the melting temperature (Tm). As described in Example 1, NYRTPH₆ (SEQ ID NO: 105) increases the thermostability relative to NYRTH₆ (SEQ ID NO: 104) for both adnectins: by 4° C. for the PCSK9 adnectin and by 6° C. for the myostatin adnectin. In addition, the presence of a second proline provides a stabilizing effect that is similar to that provided by a single proline.

Thus, the presence of one or two C-terminal prolines enhances the thermostability of adnectins relative to the same molecule without the C-terminal proline(s).

### Example 4: Addition of a C-terminal Proline Enhances Solubility

This Example demonstrates that the presence of a C-terminal proline enhances the solubility of a tandem adnectin relative to the same tandem adnectin without the C-terminal proline.

A tandem adnectin, i.e., two adnectins each binding to a different target linked by a linker, and containing either an NYRTE (SEQ ID NO: 107)C-terminus or an NYRTP (SEQ ID NO: 108)C-terminus were expressed in E. coli BLR cells and fermented at 30° C. The titer of the tandem without the proline was 1.2 g/L soluble protein and 2.8 g/L of total protein, indicating that the expressed protein was 43% soluble. The titer of the tandem with the proline was 2.56 g/L soluble and 2.61 g/L total. Even after renaturing the insoluble fraction of the tandem without the proline, and obtaining a solution that was 98% pure and monomeric at 2.5 mg/mL, this protein composition did not show significant binding to its target, suggesting that it may not be properly refolded.

Thus, considering that the only difference between the two proteins was the presence of an E versus a P at the C-terminus, the results suggests that the presence of a proline provides enhanced solubility of the tandem.

The entire disclosure of each document cited (including patents, patent applications, journal articles, abstracts, laboratory manuals, books, GENBANK® Accession numbers, SWISS-PROT® Accession numbers, or other disclosures) in the Background, Detailed Description, Brief Description of the Drawings, and Examples is hereby incorporated herein by reference in their entirety.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

1-17. (canceled)

18. A nucleic acid encoding a multivalent fibronectin based scaffold (FBS) protein comprising two or more ¹⁰Fn3 domains, wherein each of the ¹⁰Fn3 domains comprises an amino acid sequence that is at least 70% identical to SEQ ID NO: 1, and wherein (i) the C-terminal amino acid at the position corresponding to position 94 of SEQ ID NO: 1 of at least one ¹⁰Fn3 domain is covalently linked through a peptide bond to a moiety consisting of the amino acid sequence PmXn, wherein P is proline, X is any amino acid, m is an integer that is at least 1 and n is 0 or an integer that is at least 1; and
(ii) the PmXn moiety provides an enhanced property to the multivalent FBS protein relative to the FBS protein that is not linked to the PmXn moiety.

19. The nucleic acid of claim 18, wherein the moiety covalently linked to the at least one ¹⁰Fn3 domain consists of PmXn, wherein n is 1-10.

20. The nucleic acid of claim 18 wherein the moiety covalently linked to the at least one ¹⁰Fn3 domain consists of PmXn, wherein n is 1-5.

21. The nucleic acid of claim 18, wherein the PmXn moiety covalently linked to the at least one ¹⁰Fn3 domain consists of PI, PC, PID, PIE, PIDK (SEQ ID NO: 61), PIEK (SEQ ID NO: 63), PIDKP (SEQ ID NO: 65), PIEKP (SEQ ID NO: 67), PIDKPS (SEQ ID NO: 69), PIEKPS (SEQ ID NO: 71), PIDKPC (SEQ ID NO: 73), PIEKPC (SEQ ID NO: 75), PIDKPSQ (SEQ ID NO: 77), PIEKPSQ (SEQ ID NO: 79), PIDKPCQ (SEQ ID NO: 81), PIEKPCQ (SEQ ID NO: 83), PHHHHHH (SEQ ID NO: 87) or PCHHHHHH (SEQ ID NO: 86).

22. The nucleic acid of claim 18, wherein each of the least ¹⁰Fn3 domains comprises the amino acid sequence LEVVAA (X)uLLISW (X) vYRITY (X)w FTV (X) xATISGL (X)y YTITVY A (X) ZISINYRT (SEQ ID NO: 16),
wherein (X)ᵤ, (X)ᵥ, (X)_{w}, (X)ₓ, (X)_{y} and (X) z consist of the wild-type amino acid sequence (SEQ ID NO: 1) or comprise at least one amino acid difference with the corresponding wild-type sequence.

23. The nucleic acid of claim 18, wherein PmXn is P or PC.

24. The nucleic acid of claim 18, wherein at least one X of PmXn is a cysteine.

25. The nucleic acid of claim 24, wherein the cysteine is conjugated to a heterologous moiety.

26. The nucleic acid of claim 25, wherein the heterologous molecule is a detectable moiety.

27. The nucleic acid of claim 25, wherein the heterologous molecule is a drug moiety and the drug moiety and the multivalent FBS protein form an FBS-drug conjugate.

28. The nucleic acid of claim 18, wherein the enhanced property conferred by the PmXn moiety is enhanced stability.

29. The nucleic acid of claim 28, wherein enhanced stability is an increase in Tm of at least ¹⁰C., 2° C., 3° C., 4° C., 5° C. or more.

30. The nucleic acid of claim 23, wherein PmXn is PC.

31. A vector comprising the nucleic acid of claim 18.

32. A host cell comprising the vector of claim 31.

33. A method of producing a multivalent FBS protein, comprising the steps of:
(a) culturing the host cell of claim 32 under conditions suitable to express the FBS protein; and
(b) isolating the FBS protein.

34. An isolated FBS protein that specifically binds to a target, wherein the FBS is a non-naturally occurring FBS, and wherein the FBS is linked at its C-terminus to a moiety consisting of the amino acid sequence PmXn, wherein P is proline, X is any amino acid, m is an integer that is at least 1 and n is 0 or an integer that is at least 1, and wherein the PmXn moiety provides an enhanced property to the FBS protein relative to the FBS protein that is not linked to the PmXn moiety.

35. A method of producing the FBS protein of claim 34, comprising the steps of:
(a) culturing a host cell comprising a nucleic acid encoding the FBS protein under conditions suitable to express the FBS protein; and
(b) isolating the FBS protein;
wherein the FBS is linked at its C-terminus to a moiety consisting of the amino acid sequence PmXn, wherein P is proline, X is any amino acid, m is an integer that is at least 1 and n is 0 or an integer that is at least 1, and wherein the PmXn moiety provides an enhanced property to the FBS protein relative to the FBS protein that is not linked to the PmXn moiety.

36. A method of producing an FBS protein drug conjugate, the method comprising covalently linking a drug moiety to an FBS protein,
wherein the FBS is linked at its C-terminus to a moiety consisting of the amino acid sequence PmXn, wherein P is proline, X is any amino acid, m is an integer that is at least 1 and n is 0 or an integer that is at least 1, and wherein the PmXn moiety provides an enhanced property to the FBS protein relative to the FBS protein that is not linked to the PmXn moiety, and
wherein the drug moiety is linked to the PmXn moiety of the FBS protein.

37. The method of claim 35, wherein the FBS protein comprises a ¹⁰Fn3 domain, wherein the ¹⁰Fn3 comprises an amino acid sequence that is at least 70% identical to SEQ ID NO:
1, and wherein the C-terminal amino acid at the position corresponding to position 94 of SEQ ID NO: 1 is covalently linked through a peptide bond to a moiety consisting of the amino acid sequence PmXn.

38. The method of claim 37, wherein:
(a) the PmXn moiety consists of P (m is 1 and n is 0);
(b) the PmXn moiety consists of PP (m is 2 and n is 0);
(c) the PmXn moiety consists of PmXn, wherein n is 1-150;
(d) the PmXn moiety consists of PmXn, wherein n is 1-10;
(e) the PmXn moiety consists of PmXn, wherein n is 1-5; or
(f) the PmXn moiety consists of PI, PC, PID, PIE, PIDK (SEQ ID NO: 61), PIEK (SEQ ID NO: 63), PIDKP (SEQ ID NO: 65), PIEKP (SEQ ID NO: 67), PIDKPS (SEQ ID NO: 69), PIEKPS (SEQ ID NO: 71), PIDKPC (SEQ ID NO: 73), PIEKPC (SEQ ID NO: 75), PIDKPSQ (SEQ ID NO: 77), PIEKPSQ (SEQ ID NO: 79), PIDKPCQ (SEQ ID NO: 81), PIEKPCQ (SEQ ID NO: 83), PHHHHHH (SEQ ID NO: 85) or PCHHHHHH (SEQ ID NO: 87).

39. The method of claim 37, wherein the FBS protein comprises the amino acid sequence LEVVAA (X)uLLISW (X) vYRITY (X)wFTV (X) xATISGL (X)yYTITVY A (X) ZISINYRT (SEQ ID NO: 16), wherein (X)u, (X)v, (X)w, (X)x, (X)y and (X) z consist of the wild-type amino acid sequence (SEQ ID NO: 1) or comprise at least one amino acid difference with the corresponding wild-type sequence.

40. The method of claim 37, wherein PmXn moiety is P or PC.

41. The method of claim 37, wherein at least one X of the PmXn moiety is a cysteine.

42. The method of claim 41, wherein the cysteine of the PmXn moiety is conjugated to the drug moiety.

43. The method of claim 35, wherein the enhanced property conferred by the PmXn moiety is enhanced stability.

44. The method of claim 41, wherein the PmXn moiety is PmCXn or PmXn1CXn2, wherein C is cysteine and n1 and n2 are independently 0 or an integer that is at least 1.

45. The method of claim 44, wherein the cysteine of the PmXn moiety is conjugated to the drug moiety.


SEQUENCE LISTING (130 sequences)

SEQ ID NO: 1 - protein, 94 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 2 - protein, 93 aa, Homo sapiens
SDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 3 - protein, 92 aa, Homo sapiens
DVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 4 - protein, 91 aa, Homo sapiens
VPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 5 - protein, 90 aa, Homo sapiens
PRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 6 - protein, 89 aa, Homo sapiens
RDLEVVAATPTSLLISWDAPAVTVRYYRITYGETGGNSPVQEFTVPGSKSTATISGLKPGVDYTITVYAVTGRGDSPASSKPISINYRT

SEQ ID NO: 7 - protein, 88 aa, Homo sapiens
DLEVVAATPTSLLISWDAPAVTVRYYRITYGETGGNSPVQEFTVPGSKSTATISGLKPGVDYTITVYAVTGRGDSPASSKPISINYRT

SEQ ID NO: 8 - protein, 87 aa, Homo sapiens
LEVVAATPTSLLISWDAPAVTVRYYRITYGETGGNSPVQEFTVPGSKSTATISGLKPGVDYTITVYAVTGRGDSPASSKPISINYRT

SEQ ID NO: 9 - protein, 166 aa, synthetic construct
  REGION 1..166: note = Synthetic polypeptide
  VARIANT 14..33: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 39..58: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 64..83: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 87..106: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 113..132: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 140..159: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
VSDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 10 - protein, 165 aa, synthetic construct
  REGION 1..165: note = Synthetic polypeptide
  VARIANT 13..32: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 38..57: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 63..82: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 86..105: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 112..131: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 139..158: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
SDVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 11 - protein, 164 aa, synthetic construct
  REGION 1..164: note = Synthetic polypeptide
  VARIANT 12..31: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 37..56: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 62..81: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 85..104: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 111..130: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 138..157: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
DVPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 12 - protein, 163 aa, synthetic construct
  REGION 1..163: note = Synthetic polypeptide
  VARIANT 11..30: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 36..55: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 61..80: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 84..103: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 110..129: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 137..156: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
VPRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 13 - protein, 162 aa, synthetic construct
  REGION 1..162: note = Synthetic polypeptide
  VARIANT 10..29: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 35..54: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 60..79: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 83..102: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 109..128: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 136..155: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
PRDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 14 - protein, 161 aa, synthetic construct
  REGION 1..161: note = Synthetic polypeptide
  VARIANT 9..28: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 34..53: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 59..78: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 82..101: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 108..127: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 135..154: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
RDLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 15 - protein, 160 aa, synthetic construct
  REGION 1..160: note = Synthetic polypeptide
  VARIANT 8..27: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 33..52: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 58..77: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 81..100: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 107..126: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 134..153: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
DLEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 16 - protein, 159 aa, synthetic construct
  REGION 1..159: note = Synthetic polypeptide
  VARIANT 7..26: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 32..51: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 57..76: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 80..99: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 106..125: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 133..152: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
LEVVAAXXXXXXXXXXXXXXXXXXXXLLISWXXXXXXXXXXXXXXXXXXXXYRITYXXXXXXXXXXXXXXXXXXXXFTVXXXXXXXXXXXXXXXXXXXXATISGLXXXXXXXXXXXXXXXXXXXXYTITVYAXXXXXXXXXXXXXXXXXXXXISINYRT

SEQ ID NO: 17 - protein, 87 aa, Homo sapiens; identical to SEQ ID NO: 8

SEQ ID NO: 18 - protein, 159 aa, synthetic construct; identical to SEQ ID NO: 16
  REGION 1..159: note = Synthetic polypeptide
  VARIANT 7..26: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 32..51: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 57..76: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 80..99: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 106..125: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent
  VARIANT 133..152: note = Any amino acid and this region may encompass 2-20, 2-15, 2-10,2-8, 5-20, 5-15, 5-10, 5-8, 6-20, 6-15, 6-10, 6-8, 2-7, 5-7, or6-7 residues, wherein some positions may be absent

SEQ ID NO: 19 - protein, 7 aa, Homo sapiens
VSDVPRD

SEQ ID NO: 20 - protein, 6 aa, Homo sapiens
SDVPRD

SEQ ID NO: 21 - protein, 5 aa, Homo sapiens
DVPRD

SEQ ID NO: 22 - protein, 4 aa, Homo sapiens
VPRD

SEQ ID NO: 23 - protein, 101 aa, synthetic construct
  REGION 1..101: note = Synthetic polypeptide
VSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQ

SEQ ID NO: 24 - protein, 10 aa, synthetic construct
  REGION 1..10: note = Synthetic peptide
MGVSDVPRDL

SEQ ID NO: 25 - protein, 9 aa, synthetic construct
  REGION 1..9: note = Synthetic peptide
GVSDVPRDL

SEQ ID NO: 26 - protein, 9 aa, synthetic construct
  REGION 1..9: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..9: note = Variant residues given in the sequence have no preference with respect to those in the annotations for variant positions"
  VARIANT 2: note = /replace="Met" or " "
MGSDVPRDL

SEQ ID NO: 27 - protein, 8 aa, synthetic construct
  REGION 1..8: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..8: note = Variant residues given in the sequence have no preference with respect to those in the annotations for variant positions"
  VARIANT 2: note = /replace="Met" or " "
MGDVPRDL

SEQ ID NO: 28 - protein, 7 aa, synthetic construct
  REGION 1..7: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..7: note = Variant residues given in the sequence have no preference with respect to those in the annotations for variant positions"
  VARIANT 2: note = /replace="Met" or " "
MGVPRDL

SEQ ID NO: 29 - protein, 6 aa, synthetic construct
  REGION 1..6: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..6: note = Variant residues given in the sequence have no preference with respect to those in the annotations for variant positions"
  VARIANT 2: note = /replace="Met" or " "
MGPRDL

SEQ ID NO: 30 - protein, 5 aa, synthetic construct
  REGION 1..5: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..5: note = Variant residues given in the sequence have no preference with respect to those in the annotations for variant positions"
  VARIANT 2: note = /replace="Met" or " "
MGRDL

SEQ ID NO: 31 - protein, 4 aa, synthetic construct
  REGION 1..4: note = Synthetic peptide
  VARIANT 1: note = /replace="Gly" or " "
  VARIANT 1..2: note = When only 1 position is present, the position may be Met or Gly.When two positions are present, the positions are Met-Gly. See specification for further explanation."
  REGION 1..4: note = Variant residues given in the sequence have no preference with respect to those in the annotations for variant positions"
  VARIANT 2: note = /replace="Met" or " "
MGDL

SEQ ID NO: 32 - protein, 6 aa, synthetic construct
  REGION 1..6: note = Synthetic peptide
MASTSG

SEQ ID NO: 33 - protein, 91 aa, synthetic construct
  REGION 1..91: note = Synthetic polypeptide
MPAPTDLRFTNETPSSLLISWTPPRVQITGYIIRYGPVGSDGRVKEFTVPPSVSSATITGLKPGTEYTISVIALKDNQESEPLRGRVTTGG

SEQ ID NO: 34 - protein, 4 aa, synthetic construct
  REGION 1..4: note = Synthetic peptide
TPSS

SEQ ID NO: 35 - protein, 7 aa, synthetic construct
  REGION 1..7: note = Synthetic peptide
TPPRVQI

SEQ ID NO: 36 - protein, 6 aa, synthetic construct
  REGION 1..6: note = Synthetic peptide
VGSDGR

SEQ ID NO: 37 - protein, 4 aa, synthetic construct
  REGION 1..4: note = Synthetic peptide
PSVS

SEQ ID NO: 38 - protein, 5 aa, synthetic construct
  REGION 1..5: note = Synthetic peptide
GLKPG

SEQ ID NO: 39 - protein, 8 aa, synthetic construct
  REGION 1..8: note = Synthetic peptide
KDNQESEP

SEQ ID NO: 40 - protein, 89 aa, synthetic construct
  REGION 1..89: note = Synthetic polypeptide
LDAPTDLQVTNVTDTSITVSWTPPSATITGYRITYTPSNGPGEPKELTVPPSSTSVTITGITPGVEYVVSVYALKDNQESPPLVGTCTT

SEQ ID NO: 41 - protein, 89 aa, synthetic construct
  REGION 1..89: note = Synthetic polypeptide
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 42 - protein, 4 aa, synthetic construct
  REGION 1..4: note = Synthetic peptide
TEDS

SEQ ID NO: 43 - protein, 7 aa, synthetic construct
  REGION 1..7: note = Synthetic peptide
TAPDAAF

SEQ ID NO: 44 - protein, 6 aa, synthetic construct
  REGION 1..6: note = Synthetic peptide
SEKVGE

SEQ ID NO: 45 - protein, 4 aa, synthetic construct
  REGION 1..4: note = Synthetic peptide
GSER

SEQ ID NO: 46 - protein, 5 aa, synthetic construct
  REGION 1..5: note = Synthetic peptide
GLKPG

SEQ ID NO: 47 - protein, 7 aa, synthetic construct
  REGION 1..7: note = Synthetic peptide
KGGHRSN

SEQ ID NO: 48 - protein, 7 aa, synthetic construct
  REGION 1..7: note = Synthetic peptide
GPGPGPG

SEQ ID NO: 49 - protein, 11 aa, synthetic construct
  REGION 1..11: note = Synthetic peptide
GPGPGPGPGPG

SEQ ID NO: 50 - protein, 6 aa, synthetic construct
  REGION 1..6: note = Synthetic peptide
PAPAPA

SEQ ID NO: 51 - protein, 12 aa, synthetic construct
  REGION 1..12: note = Synthetic peptide
PAPAPAPAPAPA

SEQ ID NO: 52 - protein, 18 aa, synthetic construct
  REGION 1..18: note = Synthetic peptide
PAPAPAPAPAPAPAPAPA

SEQ ID NO: 53 - protein, 10 aa, synthetic construct
  REGION 1..10: note = Synthetic peptide
GSGSGSGSGS

SEQ ID NO: 54 - protein, 12 aa, synthetic construct
  REGION 1..12: note = Synthetic peptide
GSGSGSGSGSGS

SEQ ID NO: 55 - protein, 20 aa, synthetic construct
  REGION 1..20: note = Synthetic peptide
GSGSGSGSGSGSGSGSGSGS

SEQ ID NO: 56 - protein, 20 aa, synthetic construct
  REGION 1..20: note = Synthetic peptide
GGGGSGGGGSGGGGSGGGGS

SEQ ID NO: 57 - protein, 25 aa, synthetic construct
  REGION 1..25: note = Synthetic peptide
GGGGSGGGGSGGGGSGGGGSGGGGS

SEQ ID NO: 58 - protein, 15 aa, synthetic construct
  REGION 1..15: note = Synthetic peptide
GGGGSGGGGSGGGSG

SEQ ID NO: 59 - protein, 93 aa, synthetic construct
  REGION 1..93: note = Synthetic polypeptide
  VARIANT 88..90: note = This region may encompass 1-3 "Pro" residues"
  REGION 88..93: note = See specification as filed for detailed description ofsubstitutions and preferred embodiments"
  VARIANT 91..93: note = Any amino acid
  VARIANT 91..93: note = This region may encompass 1-3 residues"
LEVVAATPTSLLISWDAPAVTVRYYRITYGETGGNSPVQEFTVPGSKSTATISGLKPGVDYTITVYAVTGRGDSPASSKPISINYRTPPPXXX

SEQ ID NO: 60 - protein, 100 aa, synthetic construct
  REGION 1..100: note = Synthetic polypeptide
  VARIANT 95..97: note = This region may encompass 1-3 "Pro" residues"
  REGION 95..100: note = See specification as filed for detailed description ofsubstitutions and preferred embodiments"
  VARIANT 98..100: note = Any amino acid
  VARIANT 98..100: note = This region may encompass 1-3 residues"
VSDVPRDLEVVAATPTSLLISWDAPAVTVRYYRITYGETGGNSPVQEFTVPGSKSTATISGLKPGVDYTITVYAVTGRGDSPASSKPISINYRTPPPXXX

SEQ ID NO: 61 - protein, 4 aa, synthetic construct
  REGION 1..4: note = Synthetic peptide
PIDK

SEQ ID NO: 62 - protein, 5 aa, synthetic construct
  REGION 1..5: note = Synthetic peptide
PPIDK

SEQ ID NO: 63 - protein, 4 aa, synthetic construct
  REGION 1..4: note = Synthetic peptide
PIEK

SEQ ID NO: 64 - protein, 5 aa, synthetic construct
  REGION 1..5: note = Synthetic peptide
PPIEK

SEQ ID NO: 65 - protein, 5 aa, synthetic construct
  REGION 1..5: note = Synthetic peptide
PIDKP

SEQ ID NO: 66 - protein, 6 aa, synthetic construct
  REGION 1..6: note = Synthetic peptide
PPIDKP

SEQ ID NO: 67 - protein, 5 aa, synthetic construct
  REGION 1..5: note = Synthetic peptide
PIEKP

SEQ ID NO: 68 - protein, 6 aa, synthetic construct
  REGION 1..6: note = Synthetic peptide
PPIEKP

SEQ ID NO: 69 - protein, 6 aa, synthetic construct
  REGION 1..6: note = Synthetic peptide
PIDKPS

SEQ ID NO: 70 - protein, 7 aa, synthetic construct
  REGION 1..7: note = Synthetic peptide
PPIDKPS

SEQ ID NO: 71 - protein, 6 aa, synthetic construct
  REGION 1..6: note = Synthetic peptide
PIEKPS

SEQ ID NO: 72 - protein, 7 aa, synthetic construct
  REGION 1..7: note = Synthetic peptide
PPIEKPS

SEQ ID NO: 73 - protein, 6 aa, synthetic construct
  REGION 1..6: note = Synthetic peptide
PIDKPC

SEQ ID NO: 74 - protein, 7 aa, synthetic construct
  REGION 1..7: note = Synthetic peptide
PPIDKPC

SEQ ID NO: 75 - protein, 6 aa, synthetic construct
  REGION 1..6: note = Synthetic peptide
PIEKPC

SEQ ID NO: 76 - protein, 7 aa, synthetic construct
  REGION 1..7: note = Synthetic peptide
PPIEKPC

SEQ ID NO: 77 - protein, 7 aa, synthetic construct
  REGION 1..7: note = Synthetic peptide
PIDKPSQ

SEQ ID NO: 78 - protein, 8 aa, synthetic construct
  REGION 1..8: note = Synthetic peptide
PPIDKPSQ

SEQ ID NO: 79 - protein, 7 aa, synthetic construct
  REGION 1..7: note = Synthetic peptide
PIEKPSQ

SEQ ID NO: 80 - protein, 8 aa, synthetic construct
  REGION 1..8: note = Synthetic peptide
PPIEKPSQ

SEQ ID NO: 81 - protein, 7 aa, synthetic construct
  REGION 1..7: note = Synthetic peptide
PIDKPCQ

SEQ ID NO: 82 - protein, 8 aa, synthetic construct
  REGION 1..8: note = Synthetic peptide
PPIDKPCQ

SEQ ID NO: 83 - protein, 7 aa, synthetic construct
  REGION 1..7: note = Synthetic peptide
PIEKPCQ

SEQ ID NO: 84 - protein, 8 aa, synthetic construct
  REGION 1..8: note = Synthetic peptide
PPIEKPCQ

SEQ ID NO: 85 - protein, 7 aa, synthetic construct
  REGION 1..7: note = Synthetic peptide
PHHHHHH

SEQ ID NO: 86 - protein, 8 aa, synthetic construct
  REGION 1..8: note = Synthetic peptide
PPHHHHHH

SEQ ID NO: 87 - protein, 8 aa, synthetic construct
  REGION 1..8: note = Synthetic peptide
PCHHHHHH

SEQ ID NO: 88 - protein, 9 aa, synthetic construct
  REGION 1..9: note = Synthetic peptide
PPCHHHHHH

SEQ ID NO: 89 - protein, 5 aa, synthetic construct
  REGION 1..5: note = Synthetic peptide
PVGVV

SEQ ID NO: 90 - protein, 184 aa, Homo sapiens
VSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVT

SEQ ID NO: 91 - protein, 9 aa, Homo sapiens
RTEIDKPSQ

SEQ ID NO: 92 - protein, 15 aa, synthetic construct
  REGION 1..15: note = Synthetic peptide
RTEIEKPSQHHHHHH

SEQ ID NO: 93 - protein, 9 aa, synthetic construct
  REGION 1..9: note = Synthetic peptide
RTPHHHHHH

SEQ ID NO: 94 - protein, 4 aa, synthetic construct
  REGION 1..4: note = Synthetic peptide
RTEC

SEQ ID NO: 95 - protein, 4 aa, synthetic construct
  REGION 1..4: note = Synthetic peptide
RTPC

SEQ ID NO: 96 - protein, 110 aa, synthetic construct
  REGION 1..110: note = Synthetic polypeptide
MGVSDVPRDLEVVAATPTSLLISWDAPAEGYGYYRITYGETGGNSPVQEFTVPVSKGTATISGLKPGVDYTITVYAVEFDFPGAGYYHRPISINYRTEIDKPSQHHHHHH

SEQ ID NO: 97 - protein, 104 aa, synthetic construct
  REGION 1..104: note = Synthetic polypeptide
MGVSDVPRDLEVVAATPTSLLISWDAPAEGYGYYRITYGETGGNSPVQEFTVPVSKGTATISGLKPGVDYTITVYAVEFDFPGAGYYHRPISINYRTPHHHHHH

SEQ ID NO: 98 - protein, 104 aa, synthetic construct
  REGION 1..104: note = Synthetic polypeptide
MGVSDVPRDLEVVAATPTSLLISWTLPHAGRAHYYRITYGETGGNSPVQEFTVPGRGVTATISGLKPGVDYTITVYAVTVTTTKVIHYKPISINYRTPHHHHHH

SEQ ID NO: 99 - protein, 99 aa, synthetic construct
  REGION 1..99: note = Synthetic polypeptide
MGVSDVPRDLEVVAATPTSLLISWDAPAEGYGYYRITYGETGGNSPVQEFTVPVSKGTATISGLKPGVDYTITVYAVEFDFPGAGYYHRPISINYRTEC

SEQ ID NO: 100 - protein, 99 aa, synthetic construct
  REGION 1..99: note = Synthetic polypeptide
MGVSDVPRDLEVVAATPTSLLISWDAPAEGYGYYRITYGETGGNSPVQEFTVPVSKGTATISGLKPGVDYTITVYAVEFDFPGAGYYHRPISINYRTPC

SEQ ID NO: 101 - protein, 11 aa, synthetic construct
  REGION 1..11: note = Synthetic peptide
NYRTEIEKPCQ

SEQ ID NO: 102 - protein, 6 aa, synthetic construct
  REGION 1..6: note = Synthetic peptide
NYRTPC

SEQ ID NO: 103 - protein, 17 aa, synthetic construct
  REGION 1..17: note = Synthetic peptide
NYRTEIEKPSQHHHHHH

SEQ ID NO: 104 - protein, 10 aa, synthetic construct
  REGION 1..10: note = Synthetic peptide
NYRTHHHHHH

SEQ ID NO: 105 - protein, 11 aa, synthetic construct
  REGION 1..11: note = Synthetic peptide
NYRTPHHHHHH

SEQ ID NO: 106 - protein, 12 aa, synthetic construct
  REGION 1..12: note = Synthetic peptide
NYRTPPHHHHHH

SEQ ID NO: 107 - protein, 5 aa, synthetic construct
  REGION 1..5: note = Synthetic peptide
NYRTE

SEQ ID NO: 108 - protein, 5 aa, synthetic construct
  REGION 1..5: note = Synthetic peptide
NYRTP

SEQ ID NO: 109-110 - skipped (2 entries)

SEQ ID NO: 111 - protein, 15 aa, synthetic construct
  REGION 1..15: note = Synthetic peptide
GGGGSGGGGSGGGGS

SEQ ID NO: 112 - protein, 110 aa, synthetic construct
  REGION 1..110: note = Synthetic polypeptide
MGVSDVPRDLEVVAATPTSLLISWTLPHAGRAHYYRITYGETGGNSPVQEFTVPGRGVTATISGLKPGVDYTITVYAVTVTTTKVIHYKPISINYRTEIDKPSQHHHHHH

SEQ ID NO: 113 - protein, 7 aa, synthetic construct
  REGION 1..7: note = Synthetic peptide
EIDKPSQ

SEQ ID NO: 114 - protein, 4 aa, synthetic construct
  REGION 1..4: note = Synthetic peptide
PPID

SEQ ID NO: 115 - protein, 4 aa, synthetic construct
  REGION 1..4: note = Synthetic peptide
PPIE

SEQ ID NO: 116 - protein, 6 aa, synthetic construct
  REGION 1..6: note = Synthetic 6xHis tag"
HHHHHH

SEQ ID NO: 117-127 - skipped (11 entries)

SEQ ID NO: 128 - protein, 91 aa, Homo sapiens
RDLEVVAATPTSLLISWDAPAVTVRYYRITYGETGGNSPVQEFTVPGSKSTATISGLKPGVDYTITVYAVTGRGDSPASSKPISINYRTEI

SEQ ID NO: 129 - skipped

SEQ ID NO: 130 - protein, 6 aa, synthetic construct
  REGION 1..6: note = Synthetic peptide
  REGION 1..6: note = Variant residues given in the sequence have no preference with respect to those in the annotations for variant positions"
  VARIANT 2..3: note = /replace=" "
  VARIANT 5..6: note = /replace=" "
PPPCCC